(19) United States
(12) Patent Application Publication
(10) Pub. No.: US 2022/0228961 A1
(43) Pub. Date: Jul. 21, 2022

(54) METHOD FOR MEASURING RHEOLOGICAL PROPERTY OF DRILLING FLUID BY USING CURVED PIPE ON SITE

(71) Applicant: Vertechs Petroleum Technology Innovation & Equipment Manufacturing Co.,LTD, Chengdu (CN)

(72) Inventors: Dengpan XIE, Chengdu (CN); Qijun ZENG, Chengdu (CN)

(73) Assignee: Vertechs Petroleum Technology Innovation & Equipment Manufacturing Co.,LTD, Chengdu (CN)

(21) Appl. No.: 17/709,467

(22) Filed: Mar. 31, 2022

Related U.S. Application Data
(63) Continuation of application No. PCT/CN2020/138476, filed on Dec. 23, 2020.

(51) Int. Cl. G01N 11/14; G01N 33/28

(52) U.S. Cl. CPC G01N 11/14; G01N 2011/0006; G01N 33/2823

(57) ABSTRACT

A method for measuring a rheological property of a drilling fluid by using a curved pipe on site includes: step 1: deriving relationship constants between friction coefficients of a drilling fluid through offline checking; step 2: calculating Rₑᵢ according to f_{ci}; step 3: calculating an actual shear stress τwᵢ and a shear rate γᵢ of the drilling fluid in the on-site curved pipe according to the relationship constants and Rₑᵢ; step 4: establishing a plurality of on-site models according to τwᵢ and γᵢ; step 5: determining an optimal on-site model according to correlations between τwᵢ and predicted shear stresses of the plurality of on-site models; and step 6: performing on-site measurement on the rheological property of the drilling fluid according to the optimal on-site model. The method avoids inaccurate rheological measurement due to different types of drilling fluids and improves the measurement accuracy for different types of drilling fluids.

### CROSS REFERENCE TO THE RELATED APPLICATIONS

This application is the continuation application of International Application No. PCT/CN2020/138476, filed on Dec. 23, 2020, the entire contents of which are incorporated herein by reference.

### TECHNICAL FIELD

The present invention relates to the field of oil well construction and, in particular, to a method for measuring a rheological property of a drilling fluid by using a curved pipe on site.

### BACKGROUND

The properties of the drilling fluid (mud) play a crucial role in optimizing drilling operations. Among them, the density and rheological properties of the drilling fluid play a significant role in the optimal management of wellbore pressure. Therefore, it is necessary to carry out accurate measurement of the density and rheological properties of the drilling fluid in narrow-window drilling operations, especially in advanced drilling techniques, including managed pressure drilling (MPD) and dual gradient drilling (DGD).

At present, the rheological properties of the drilling fluid are mainly measured by a rotation method and a pipe flow method. In the pipe flow method, the online rheological measurement device uses a straight pipe for measurement. However, due to its large size, it requires significant changes to the site space, resulting in greatly limited on-site applications and poor measurement accuracy.

### SUMMARY

A technical problem to be solved by the present invention is to provide a method for measuring a rheological property of a drilling fluid by using a curved pipe on site, so as to improve the accuracy of the rheological measurement of the drilling fluid.

To solve the above technical problem, the present invention adopts the following technical solution: a method for measuring a rheological property of a drilling fluid by using a curved pipe on site, including the following steps:

step 1: deriving relationship constants between friction coefficients of a drilling fluid through offline checking;

step 2: calculating a Reynolds number Rₑᵢ of the drilling fluid in an on-site curved pipe according to a friction coefficient f_{ci} of the drilling fluid in the on-site curved pipe;

step 3: calculating an actual shear stress τwᵢ of the drilling fluid in the on-site curved pipe according to the relationship constants between the friction coefficients of the drilling fluid and the Reynolds number Rₑᵢ of the drilling fluid in the on-site curved pipe, where i denotes a number of times the drilling fluid flows through the on-site curved pipe, which is a positive integer not less than 2;

step 4: establishing a plurality of on-site models according to the actual shear stress τwᵢ and a shear rate γ of the drilling fluid;

step 5: determining an optimal on-site model according to correlations between the actual shear stress τwᵢ and predicted shear stresses of the plurality of on-site models; and

step 6: performing on-site measurement on the rheological property of the drilling fluid according to the optimal on-site model.

The working principle and beneficial effects of the present invention are as follows: The present invention derives the relationship constants between the friction coefficients of the drilling fluid through offline checking and can derive the relationship constants for different types of drilling fluids. The present invention avoids inaccurate rheological measurement due to different types of drilling fluids and improves the measurement accuracy for different types of drilling fluids. In addition, the present invention determines the optimal on-site model according to the correlations between the actual shear stress τwᵢ and the predicted shear stresses of the plurality of on-site models, so as to ensure the accuracy of on-site measurement of the drilling fluid.

The following improvement may be further made by the present invention based on the above technical solution.

Further, step 1 may include:

step 11: calculating a friction coefficient f_{ck} of the drilling fluid in an offline curved pipe and a friction coefficient fₛₖ of the drilling fluid in an offline straight pipe, where, k denotes a number of times the drilling fluid flows through an offline pipe, which is a positive integer not less than 2;

step 12: establishing a plurality of prediction models according to an actual friction coefficient ratio yₖ, where yₖ=f_{ck}/fₛₖ;

step 13: determining an optimal prediction model according to correlations between the actual friction coefficient ratio yₖ and predicted friction coefficient ratios of the plurality of prediction models; and

step 14: deriving the relationship constants between the friction coefficients of the drilling fluid according to the optimal prediction model.

The beneficial effects of the above further solution are as follows. The present invention measures and calculates the actual friction coefficients of the drilling fluid in offline curved and straight pipes and establishes the plurality of prediction models. The present invention determines the optimal prediction model according to the correlations between the actual friction coefficient ratio yᵢ and the predicted friction coefficient ratios of the plurality of prediction models. The present invention ensures the accuracy of selecting the prediction model. The present invention uses the relationship constants between the friction coefficients of the selected prediction model for subsequent on-site measurement on the rheological property of the drilling fluid. The present invention analyzes the correlations through the plurality of prediction models to ensure the accuracy of the relationship constants between the friction coefficients of different types of drilling fluids. Therefore, the present invention avoids the inaccuracy caused when a single relationship constant between the friction coefficients is applied to the on-site measurement of the drilling fluid.

The following improvement may be further made by the present invention based on the above technical solution.

Further, in step 11, f_{ck} may be expressed by formula (1):

$${f}_{c k} = \frac{{d}_{tc 1}}{2 {ρ}_{1} * {v}_{c k}^{2}} * \frac{Δ {P}_{c k}}{Δ {L}_{c k}} \qquad ( 1 )$$

where, d_{tc1} denotes an inner diameter of the offline curved pipe, and has a unit of m;

ρ₁ denotes a density of an offline drilling fluid, and has a unit of kg/m³;

v_{ck} denotes a flow velocity of the drilling fluid at the k-th time in the offline curved pipe, and has a unit of m/s; and

ΔP_{ck}/ΔL_{ck} denotes a measured average pressure difference in the offline curved pipe, and has a unit of kPa/m; and ΔP_{ck} denotes a total pressure difference in a pipe section with a length of ΔL_{ck}, and has a unit of kPa;

in step 1, fₛₖ may be expressed by formula (2):

$${f}_{sk} = \frac{{d}_{ts 1}}{2 {ρ}_{1} * {v}_{sk}^{2}} * \frac{Δ {P}_{sk}}{Δ {L}_{sk}} \qquad ( 2 )$$

where, dₜₛ₁ denotes an inner diameter of the offline straight pipe, and has a unit of m;

ρ₁ denotes a density of the drilling fluid, and has a unit of kg/m³;

vₛₖ denotes a flow velocity of the drilling fluid at the k-th time in the offline straight pipe, and has a unit of m/s; and

ΔPₛₖ/ΔLₛₖ denotes a measured average pressure difference in the offline straight pipe, and has a unit of kPa/m; and ΔPₛₖ denotes a total pressure difference in a pipe section with a length of ΔLₛₖ, and has a unit of kPa.

The beneficial effects of the above further solution are as follows. The present invention measures the average pressure differences of the straight pipe and the curved pipe and calculates the friction coefficient f_{ck} of the drilling fluid in the curved pipe and the friction coefficient fₛₖ thereof in the straight pipe, so as to ensure the calculation accuracy of the friction coefficients.

The following improvement may be further made by the present invention based on the above technical solution.

Further,

there may be at least three prediction models, namely:

a first prediction model:

ŷ₁ₖ=a*Dₙₖ^{b}+C (3)

a second prediction model:

$${\hat{y}}_{2 k} = 1 + \frac{a * {D}_{n k}^{b}}{70 + {D}_{n k}} \qquad ( 4 )$$

a third prediction model:

ŷ₃ₖ=1+a*(log₁₀Dₙₖ)^{b} (5)

where

ŷ₁ₖ denotes a predicted friction coefficient of the first prediction model;

ŷ₂ₖ denotes a predicted friction coefficient of the second prediction model;

ŷ₃ₖ denotes a predicted friction coefficient of the third prediction model; and

a, b and c denote the relationship constants between the friction coefficients of the drilling fluid, respectively;

where, Dₙₖ denotes a Dean number of the drilling fluid at the k-th time in the offline curved pipe, and is expressed by formula (6):

$${D}_{n k} = \frac{{ρ}_{1} * {v}_{ck} * {d}_{tc 1}}{{μ}_{1}} * \sqrt{\frac{{d}_{tc 1}}{{D}_{c 1}}} \qquad ( 6 )$$

where

μ₁ denotes a viscosity of an offline drilling fluid, and has a unit of Pa·s; and

v_{ck} denotes a flow velocity of the drilling fluid at the k-th time in the offline curved pipe, and has a unit of m/s.

The advantages of the above further solution are as follows. The present invention designs a plurality of prediction models involving the Dean number, which ensures the calculation accuracy.

The following improvement may be further made by the present invention based on the above technical solution.

Further,

step 13 may specifically include:

expressing a correlation R₁₁² between the actual friction coefficient ratio yₖ and a predicted friction coefficient ratio of the first prediction model by formula (7):

$${R}_{11}^{2} = 1 - \frac{{{\sum }_{k = 1}}^{m} {( {y}_{k} - \hat{{y}_{1 k}} )}^{2}}{{{\sum }_{k = 1}}^{m} {( {y}_{k} - \overline{y} )}^{2}} \qquad ( 7 )$$

expressing a correlation R₁₂² between the actual friction coefficient ratio yₖ and a predicted friction coefficient ratio of the second prediction model by formula (8):

$${R}_{12}^{2} = 1 - \frac{{{\sum }_{k = 1}}^{m} {( {y}_{k} - \hat{{y}_{2 k}} )}^{2}}{{{\sum }_{k = 1}}^{m} {( {y}_{k} - \overline{y} )}^{2}} \qquad ( 8 )$$

expressing a correlation R₁₃² between the actual friction coefficient ratio yₖ and a predicted friction coefficient ratio of the third prediction model by formula (9):

$${R}_{13}^{2} = 1 - \frac{{{\sum }_{k = 1}}^{m} {( {y}_{k} - \hat{{y}_{3 k}} )}^{2}}{{{\sum }_{k = 1}}^{m} {( {y}_{k} - \overline{y} )}^{2}} \qquad ( 9 )$$

comparing R₁₁², R₁₂² and R₁₃² in terms of magnitude, and selecting a prediction model with a maximum correlation as an optimal prediction model;

where

m denotes a number of samples;

yₖ denotes the actual friction coefficient ratio; and

y̅ denotes an average actual friction coefficient ratio.

The beneficial effects of the above further solution are as follows: The present invention selects the final model for offline calibration through the correlations between the actual friction coefficient ratio yᵢ and the predicted friction coefficient ratios of the prediction models, which ensures the accuracy of selecting the offline model.

The following improvement may be further made by the present invention based on the above technical solution.

Further,

in step 2, f_{ci} may be expressed by formula (10):

$${f}_{ci} = \frac{{d}_{tc 2}}{2 {ρ}_{2} * {v}_{ci}^{2}} * \frac{Δ {P}_{ci}}{Δ {L}_{ci}} \qquad ( 10 )$$

where, d_{tc2} denotes an inner diameter of the on-site curved pipe, and has a unit of m;

ρ₂ denotes a density of an on-site drilling fluid, and has a unit of kg/m³;

v_{ci} denotes a flow velocity of the drilling fluid at the i-th time in the on-site curved pipe, and has a unit of m/s; and

ΔP_{ci}/ΔL_{ci} denotes a measured average pressure difference in the on-site curved pipe, and has a unit of kPa/m; and ΔP_{ci} denotes a total pressure difference in a pipe section with a length of ΔL_{ci}, and has a unit of kPa.

The beneficial effects of the above further solution are as follows. The present invention can accurately calculate the friction coefficient f_{ci} of the drilling fluid in the on-site curved pipe.

The following improvement may be further made by the present invention based on the above technical solution.

Further,

in step 2, the Reynolds number Rₑᵢ of the drilling fluid in the on-site curved pipe may be calculated as follows:

when an offline model is the first prediction model, the Reynolds number Rₑᵢ of the drilling fluid in the on-site curved pipe satisfies formula (12):

$${f}_{ci} = \frac{16}{{R}_{ei}} ( a * {( {R}_{ei} * \sqrt{\frac{{d}_{tc 2}}{{D}_{c 2}}} )}^{b} + c ) \qquad ( 12 )$$

when the offline model is the second prediction model, the Reynolds number Rₑᵢ of the drilling fluid in the on-site curved pipe satisfies formula (13):

$${f}_{ci} = \frac{16}{{R}_{ei}} * [ 1 + \frac{a * {( {R}_{ei} * \sqrt{\frac{{d}_{tc 2}}{{D}_{c 2}}} )}^{b}}{70 + ( {R}_{ei} * \sqrt{\frac{{d}_{tc 2}}{{D}_{c 2}}} )} ] , \qquad ( 13 )$$

and

when the offline model is the third prediction model, the Reynolds number Rₑᵢ of the drilling fluid in the on-site curved pipe satisfies formula (14):

$${f}_{ci} = \frac{16}{{R}_{ei}} * [ 1 + a * {( {log}_{10} ( {R}_{ei} * \sqrt{\frac{{d}_{tc 2}}{{D}_{c 2}}} ) )}^{b} ] . \qquad ( 14 )$$

The beneficial effects of the above further solution are as follows. The present invention calculates the Reynolds number Rₑᵢ of the drilling fluid in the on-site curved pipe and adopts different calculation methods according to different offline models, thereby ensuring the accuracy of the calculation of the Reynolds number Rₑᵢ of the drilling fluid in the on-site curved pipe.

The following improvement may be further made by the present invention based on the above technical solution.

Further,

in step 3, the actual shear stress τwᵢ of the drilling fluid in the on-site curved pipe may be expressed by formula (15):

$${τ}_{w i} = \frac{8 {ρ}_{2} * {v}_{c i}^{2}}{{R}_{e i}} \qquad ( 15 )$$

where

v_{ci} denotes the flow velocity of the drilling fluid at the i-th time in the on-site curved pipe, and has a unit of m/s; and

ρ₂ denotes the density of the on-site drilling fluid, and has a unit of kg/m³;

the shear rate γᵢ of the drilling fluid is expressed by formula (16):

$${γ}_{i} = \frac{8 * {v}_{ci}}{{d}_{tc 2}} * \frac{3 * {N}_{i} + 1}{4 * {N}_{i}} \qquad ( 16 )$$

where, N is expressed by formula (17):

$${N}_{i} = \frac{d ( ln {τ}_{{w}_{i}} )}{d ( ln \frac{8 * {v}_{ci}}{{d}_{tc 2}} )} \qquad ( 17 )$$

The beneficial effects of the above further solution are as follows. The present invention ensures the calculation accuracy.

The following improvement may be further made by the present invention based on the above technical solution.

Further,

there may be at least three on-site models, namely:

a first on-site model:

τŵ₁ᵢ=YP−PV*γᵢ (18)

a second on-site model:

τŵ₂ᵢ=K*γᵢⁿ (19)

a third on-site model:

τŵ₂ᵢ=τ₀+K*γᵢⁿ (20)

where

YP denotes a yield strength of the on-site drilling fluid, and has a unit of Pa;

PV denotes a plastic viscosity of the on-site drilling fluid, and has a unit of Pa·s;

n denotes a fluidity index of the on-site drilling fluid, and is dimensionless;

K denotes a consistency coefficient of the on-site drilling fluid, and has a unit of Pa·s\hat{ }n; and

τ₀ denotes a dynamic shear stress of the on-site drilling fluid, and has a unit of Pa.

The beneficial effects of the above further solution are as follows. The present invention selects the rheological parameters through three different on-site models to ensure the optimal on-site models available for different drilling fluids.

The following improvement may be further made by the present invention based on the above technical solution.

Further,

step 5 may specifically include:

expressing a correlation R₂₁² between the actual shear stress τwᵢ and a predicted shear stress of the first on-site model by formula (21):

$${R}_{21}^{2} = 1 - \frac{{{\sum }_{i = 1}}^{m} {( τ {w}_{i} - τ {\hat{w}}_{1 i} )}^{2}}{{{\sum }_{i = 1}}^{m} {( τ {w}_{i} - \overline{τ w} )}^{2}} \qquad ( 21 )$$

expressing a correlation R₂₂² between the actual shear stress τwᵢ and a predicted shear stress of the second on-site model by formula (22):

$${R}_{22}^{2} = 1 - \frac{{{\sum }_{i = 1}}^{m} {( τ {w}_{i} - τ {\hat{w}}_{2 i} )}^{2}}{{{\sum }_{i = 1}}^{m} {( τ {w}_{i} - \overline{τ w} )}^{2}} \qquad ( 22 )$$

expressing a correlation R₂₃² between the actual shear stress τwᵢ and a predicted shear stress of the third on-site model by formula (23):

$${R}_{23}^{2} = 1 - \frac{{{\sum }_{i = 1}}^{m} {( τ {w}_{i} - τ {\hat{w}}_{3 i} )}^{2}}{{{\sum }_{i = 1}}^{m} {( τ {w}_{i} - \overline{τ w} )}^{2}} \qquad ( 23 )$$

comparing R₂₁², R₂₂² and R₂₃² in terms of magnitude, and selecting an on-site model with a maximum correlation as a final model;

where

m denotes a number of samples;

τwᵢ denotes the actual shear stress; and

τ̅w̅ denotes an average actual shear stress.

The beneficial effects of the above further solution are as follows: The present invention selects the final model for on-site measurement through the correlations between the actual shear stress τwᵢ and the predicted friction coefficient ratios of the on-site models, ensuring the accuracy of selecting the on-site model.

### BRIEF DESCRIPTION OF THE DRAWINGS

FIG. 1 is a flowchart of an on-site measurement method according to a first embodiment of the present invention; and

FIG. 2 is a flowchart of an offline checking method according to the first embodiment of the present invention.

### DETAILED DESCRIPTION OF THE EMBODIMENTS

Principles and features of the present invention are described below with reference to the drawings. The described embodiments are only used to explain the present invention, rather than to limit the scope of the present invention.

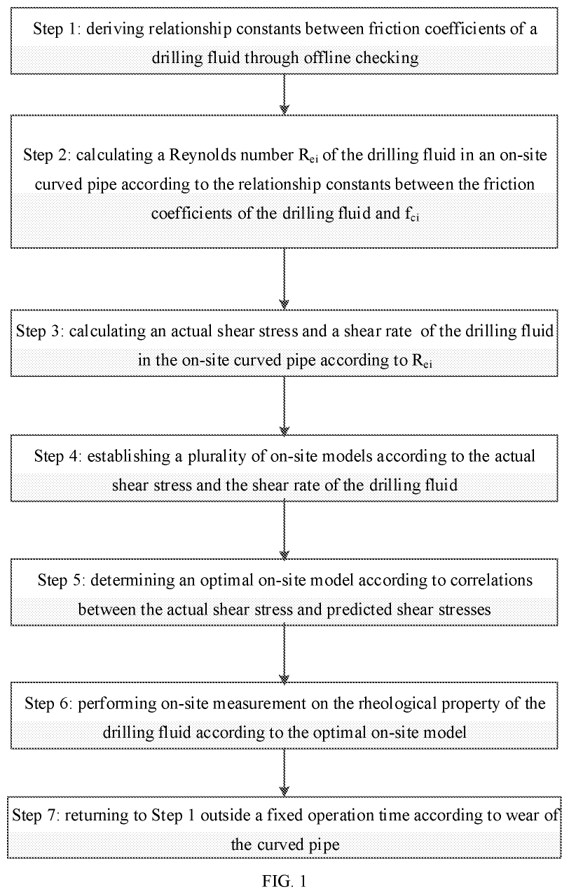

FIG. 1 is a flowchart of an on-site measurement method according to a first embodiment of the present invention.

A method for measuring a rheological property of a drilling fluid by using a curved pipe on site includes the following steps:

Step 1: Derive relationship constants between friction coefficients of a drilling fluid through offline checking.

Step 2: Calculate a Reynolds number Rₑᵢ of the drilling fluid in an on-site curved pipe according to a friction coefficient f_{ci} of the drilling fluid in the curved pipe.

Step 3: Calculate an actual shear stress τwᵢ of the drilling fluid in the on-site curved pipe according to the relationship constants between the friction coefficients of the drilling fluid and the Reynolds number Rₑᵢ of the drilling fluid in the on-site curved pipe, where i denotes a number of times the drilling fluid flows through the on-site curved pipe, which is a positive integer not less than 2.

Step 4: Establish a plurality of on-site models according to the actual shear stress τwᵢ and a shear rate γ of the drilling fluid.

Step 5: Determine an optimal on-site model according to correlations between the actual shear stress τwᵢ and predicted shear stresses of the plurality of on-site models.

Step 6: Perform on-site measurement on the rheological property of the drilling fluid according to the optimal on-site model.

The working principle and beneficial effects of the embodiment of the present invention are as follows. The present invention derives the relationship constants between the friction coefficients of the drilling fluid through offline checking and can derive the relationship constants for different types of drilling fluids. The present invention avoids inaccurate rheological measurement due to different types of drilling fluids and improves the measurement accuracy for different types of drilling fluids. In addition, the present invention determines the optimal on-site model according to the correlations between the actual shear stress τwᵢ and the predicted shear stresses of the plurality of on-site models, so as to ensure the accuracy of on-site measurement of the drilling fluid.

In this embodiment, when the on-site measurement on the rheological property of the drilling fluid is carried out by the optimal on-site model, if the friction of the curved pipe changes, Step 7 is performed. That is, according to the friction of the curved pipe, the entire method starting from Step 1 is repeated outside a fixed operation time. The fixed operation time refers to a normal operation time of the on-site measurement equipment.

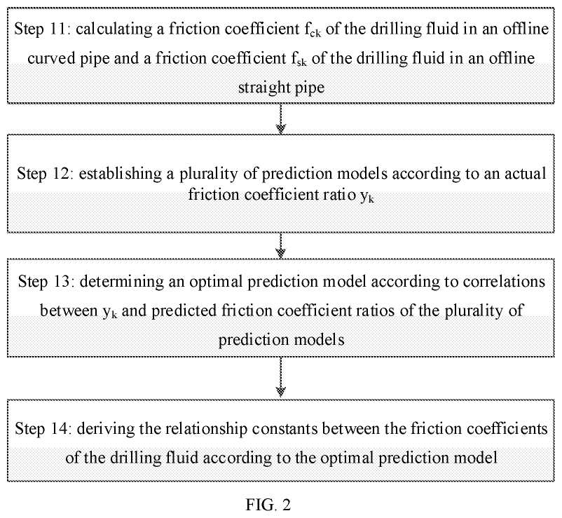

FIG. 2 is a flowchart of an offline checking method according to the embodiment of the present invention.

Step 1 includes:

Step 11: Calculate a friction coefficient f_{ck} of the drilling fluid in an offline curved pipe and a friction coefficient fₛₖ of the drilling fluid in an offline straight pipe, where, k denotes a number of times the drilling fluid flows through an offline pipe, which is a positive integer not less than 2.

Step 12: Establish a plurality of prediction models according to an actual friction coefficient ratio yₖ, where, yₖ=f_{ck}/fₛₖ.

Step 13: Determine an optimal prediction model according to correlations between the actual friction coefficient ratio yₖ and predicted friction coefficient ratios of the plurality of prediction models.

Step 14: Derive the relationship constants between the friction coefficients of the drilling fluid according to the optimal prediction model.

The present invention measures and calculates the actual friction coefficients of the drilling fluid in offline curved and straight pipes and establishes the plurality of prediction models. The present invention determines the optimal prediction model according to the correlations between the actual friction coefficient ratio yᵢ and the predicted friction coefficient ratios of the plurality of prediction models. The present invention ensures the accuracy of selecting the prediction model. The present invention uses the relationship constants between the friction coefficients of the selected prediction model for subsequent on-site measurement on the rheological property of the drilling fluid. The present invention analyzes the correlations through the plurality of prediction models to ensure the accuracy of the relationship constants between the friction coefficients of different types of drilling fluids. Therefore, the present invention avoids the inaccuracy caused when a single relationship constant between the friction coefficients is applied to the on-site measurement of the drilling fluid.

Specifically, in step 11, f_{ck} is expressed by formula (1):

$${f}_{c k} = \frac{{d}_{tc 1}}{2 {ρ}_{1} * {v}_{c k}^{2}} * \frac{Δ {P}_{c k}}{Δ {L}_{c k}} \qquad ( 1 )$$

where, d_{tc1} denotes an inner diameter of the offline curved pipe, and has a unit of m;

ρ₁ denotes a density of an offline drilling fluid, and has a unit of kg/m³;

v_{ck} denotes a flow velocity of the drilling fluid at the k-th time in the offline curved pipe, and has a unit of m/s; and

ΔP_{ck}/ΔL_{ck} denotes a measured average pressure difference in the offline curved pipe, and has a unit of kPa/m; and ΔP_{ck} denotes a total pressure difference in a pipe section with a length of ΔL_{ck}, and has a unit of kPa;

in step 1, fₛₖ is expressed by formula (2):

$${f}_{c k} = \frac{{d}_{ts 1}}{2 {ρ}_{1} * {v}_{sk}^{2}} * \frac{Δ {P}_{sk}}{Δ {L}_{sk}} \qquad ( 2 )$$

where, dₜₛ₁ denotes an inner diameter of the offline straight pipe, and has a unit of m;

ρ₁ denotes a density of the drilling fluid, and has a unit of kg/m³;

vₛₖ denotes a flow velocity of the drilling fluid at the k-th time in the offline straight pipe, and has a unit of m/s; and

ΔPₛₖ/ΔLₛₖ denotes a measured average pressure difference in the offline straight pipe, and has a unit of kPa/m; and ΔPₛₖ denotes a total pressure difference in a pipe section with a length of ΔLₛₖ, and has a unit of kPa.

where,

$${d}_{tc 1} = \sqrt{\frac{4 * {V}_{1}}{π * le {n}_{1}}} \qquad ( 24 )$$

where, V₁ denotes a total volume of the offline curved pipe, and has a unit of m³; and

len₁ denotes an length of the offline curved pipe, and has a unit of m.

In this embodiment, there are at least three prediction models, namely:

a first prediction model:

ŷ₁ₖ=a*Dₙₖ^{b}+c (3)

a second prediction model:

$${\hat{y}}_{2 k} = 1 + \frac{a * {D}_{n k}^{b}}{70 + {D}_{n k}} \qquad ( 4 )$$

a third prediction model:

ŷ₃ₖ=1+a*(log₁₀Dₙₖ)^{b} (5)

where

ŷ₁ₖ denotes a predicted friction coefficient of the first prediction model;

ŷ₂ₖ denotes a predicted friction coefficient of the second prediction model;

ŷ₃ₖ denotes a predicted friction coefficient of the third prediction model; and

a, b and c denote the relationship constants between the friction coefficients of the drilling fluid, respectively;

where, Dₙₖ denotes a Dean number of the drilling fluid at the k-th time in the offline curved pipe, and is expressed by formula (6):

$${D}_{n k} = \frac{{ρ}_{1} * {v}_{ck} * {d}_{tc 1}}{{μ}_{1}} * \sqrt{\frac{{d}_{tc 1}}{{D}_{c 1}}} \qquad ( 6 )$$

where

μ₁ denotes a viscosity of an offline drilling fluid, and has a unit of Pa·s; and

v_{ck} denotes a flow velocity of the drilling fluid at the k-th time in the offline curved pipe, and has a unit of m/s.

Step 13 specifically includes:

Express a correlation R₁₁² between the actual friction coefficient ratio yk and a predicted friction coefficient ratio of the first prediction model by formula (7):

$${R}_{11}^{2} = 1 - \frac{{{\sum }_{k = 1}}^{m} {( {y}_{k} - \hat{{y}_{1 k}} )}^{2}}{{{\sum }_{k = 1}}^{m} {( {y}_{k} - \overline{y} )}^{2}} \qquad ( 7 )$$

express a correlation R122 between the actual friction coefficient ratio yₖ and a predicted friction coefficient ratio of the second prediction model by formula (8):

$${R}_{12}^{2} = 1 - \frac{{{\sum }_{k = 1}}^{m} {( {y}_{k} - \hat{{y}_{2 k}} )}^{2}}{{{\sum }_{k = 1}}^{m} {( {y}_{k} - \overline{y} )}^{2}} \qquad ( 8 )$$

express a correlation R132 between the actual friction coefficient ratio yₖ and a predicted friction coefficient ratio of the third prediction model by formula (9):

$${R}_{13}^{2} = 1 - \frac{{{\sum }_{k = 1}}^{m} {( {y}_{k} - \hat{{y}_{3 k}} )}^{2}}{{{\sum }_{k = 1}}^{m} {( {y}_{k} - \overline{y} )}^{2}} \qquad ( 9 )$$

compare R₁₁², R₁₂² and R₁₃² in terms of magnitude, and select a prediction model with a maximum correlation as an optimal prediction model;

where

m denotes a number of samples;

yₖ denotes the actual friction coefficient ratio; and

y̅ denotes an average actual friction coefficient ratio.

In this embodiment, in step 2, f_{ci} is expressed by formula (10):

$${f}_{ci} = \frac{{d}_{t c 2}}{2 {ρ}_{2} * {v}_{ci}^{2}} * \frac{Δ {P}_{ci}}{Δ {L}_{ci}} \qquad ( 10 )$$

where, d_{tc2} denotes an inner diameter of the on-site curved pipe, and has a unit of m;

ρ₂ denotes a density of an on-site drilling fluid, and has a unit of kg/m³;

v_{ci} denotes a flow velocity of the drilling fluid at the i-th time in the on-site curved pipe, and has a unit of m/s; and

ΔP_{ci}/ΔL_{ci} denotes a measured average pressure difference in the on-site curved pipe, and has a unit of kPa/m; and ΔP_{ci} denotes a total pressure difference in a pipe section with a length of ΔL_{ci}, and has a unit of kPa; and

d_{tc2} is expressed by formula (11):

$${d}_{t c 2} = \sqrt{\frac{4 * {V}_{2}}{π * {len}_{2}}} \qquad ( 11 )$$

where, V₂ denotes a total volume of the on-site curved pipe, and has a unit of m³; and

len₂ denotes a length of the on-site curved pipe, and has a unit of m;

Specifically, in step 2, the Reynolds number Rₑᵢ of the drilling fluid in the on-site curved pipe is calculated as follows:

when an offline model is the first prediction model, the Reynolds number Rₑᵢ of the drilling fluid in the on-site curved pipe satisfies formula (12):

$${f}_{ci} = \frac{16}{{R}_{ei}} ( a * {( {R}_{ei} * \sqrt{\frac{{d}_{tc 2}}{{D}_{c 2}}} )}^{b} + c ) \qquad ( 12 )$$

when the offline model is the second prediction model, the Reynolds number Rₑᵢ of the drilling fluid in the on-site curved pipe satisfies formula (13):

$${f}_{ci} = \frac{16}{{R}_{ei}} * [ 1 + \frac{{{a}^{*} ( {R}_{ei} * \sqrt{\frac{{d}_{tc 2}}{{D}_{c 2}}} )}^{b}}{70 + ( {R}_{ei} * \sqrt{\frac{{d}_{tc 2}}{{D}_{c 2}}} )} ] \qquad ( 13 )$$

when the offline model is the third prediction model, the Reynolds number Rₑᵢ of the drilling fluid in the on-site curved pipe satisfies formula (14):

$${f}_{ci} = \frac{16}{{R}_{ei}} * [ 1 + {{a}^{*} ( {log}_{10} ( {R}_{ei} * \sqrt{\frac{{d}_{tc 2}}{{D}_{c 2}}} ) )}^{b} ] . \qquad ( 14 )$$

Specifically, in step 3, the actual shear stress τwᵢ of the drilling fluid in the on-site curved pipe is expressed by formula (15):

$${τ}_{w i} = \frac{8 {ρ}_{2} ⋆ {v}_{c i}^{2}}{{R}_{e i}} \qquad ( 15 )$$

where

v_{ci} denotes the flow velocity of the drilling fluid at the i-th time in the on-site curved pipe, and has a unit of m/s; and

ρ₂ denotes the density of the on-site drilling fluid, and has a unit of kg/m³;

the shear rate γᵢ of the drilling fluid is expressed by formula (16):

$${γ}_{i} = \frac{{8}^{*} {v}_{ci}}{{d}_{t c 2}} * \frac{{3}^{*} {N}_{i} + 1}{{4}^{*} {N}_{i}} \qquad ( 16 )$$

where, N is expressed by formula (17):

$${N}_{i} = \frac{d ( ln {τ}_{{w}_{i}} )}{d ( ln \frac{{8}^{*} {v}_{ci}}{{d}_{t c 2}} )} \qquad ( 17 )$$

In this embodiment, there are at least three on-site model, namely:

a first on-site model:

\hat{τ}w₁ᵢ=YP+PV*γᵢ (18)

a second on-site model:

\hat{τ}w₂ᵢ=K*γᵢⁿ (19)

a third on-site model:

\hat{τ}w₂ᵢ=τ₀K*γᵢⁿ (20)

where

YP denotes a yield strength of the on-site drilling fluid, and has a unit of Pa;

PV denotes a plastic viscosity of the on-site drilling fluid, and has a unit of Pa·s;

n denotes a fluidity index of the on-site drilling fluid, and is dimensionless;

K denotes a consistency coefficient of the on-site drilling fluid, and has a unit of Pa·s\hat{ }n; and

τ₀ denotes a dynamic shear stress of the on-site drilling fluid, and has a unit of Pa.

Specifically, step 5 includes:

express a correlation R₂₁² between the actual shear stress τwᵢ and a predicted shear stress of the first on-site model by formula (21):

$${R}_{21}^{2} = 1 - \frac{{\sum }_{i = 1}^{m} {( τ {w}_{i} - τ {\hat{w}}_{1 i} )}^{2}}{{\sum }_{i = 1}^{m} {( τ {w}_{i} - \overline{τw} )}^{2}} \qquad ( 21 )$$

express a correlation R₂₂² between the actual shear stress τwᵢ and a predicted shear stress of the second on-site model by formula (22):

$${R}_{22}^{2} = 1 - \frac{{\sum }_{i = 1}^{m} {( τ {w}_{i} - τ {\hat{w}}_{2 i} )}^{2}}{{\sum }_{i = 1}^{m} {( τ {w}_{i} - \overline{τw} )}^{2}} \qquad ( 22 )$$

express a correlation R₂₃² between the actual shear stress τwᵢ and a predicted shear stress of the third on-site model by formula (23):

$${R}_{23}^{2} = 1 - \frac{{\sum }_{i = 1}^{m} {( τ {w}_{i} - τ {\hat{w}}_{3 i} )}^{2}}{{\sum }_{i = 1}^{m} {( τ {w}_{i} - \overline{τw} )}^{2}} \qquad ( 23 )$$

compare R₂₁², R₂₂² and R₂₃² in terms of magnitude, and selecting an on-site model with a maximum correlation as a final model;

where

m denotes a number of samples;

τwᵢ denotes the actual shear stress; and

τ̅w̅ denotes an average actual shear stress.

The application of the first embodiment of offline checking of the present invention is described below.

In this embodiment, the offline curved pipe is a helical pipe, which has a total volume V₁=1.04 l and a length len₁=5.57476 m; the offline straight pipe has an inner diameter dₜₛ₁=0.01056 m; and a first offline drilling fluid has a density ρ₁=1,003 kg/m³.

The inner diameter of the offline curved pipe is calculated as: d_{tc1}=0.01051 m:

$${d}_{tc 1} = \sqrt{\frac{{4}^{*} {V}_{1}}{{π}^{*} {len}_{1}}} \qquad ( 4 )$$

In this embodiment, the first offline drilling fluid flows through the pipe for k=24 times.

A flow velocity vₛₖ of the first offline drilling fluid at the k-th time in a straight pipe and a flow velocity v_{ck} thereof at the k-th time in the curved pipe are shown in the table below.

An average pressure difference ΔP_{ck}/ΔL_{ck} of the offline curved pipe and an average pressure difference ΔPₛₖ/ΔLₛₖ of the offline straight pipe are also shown in the table below.

The first offline drilling fluid flows through for 24 times, and its friction coefficient f_{ck} in the offline curved pipe and friction coefficient fₛₖ in the offline straight pipe are calculated by formulas (1) and (2) and are shown in the table below.

$${f}_{c k} = \frac{{d}_{tc 1}}{2 {ρ}_{1} ⋆ {v}_{c k}^{2}} * \frac{Δ {P}_{c k}}{Δ {L}_{c k}} \qquad ( 1 )$$

$${f}_{sk} = \frac{{d}_{ts 1}}{2 {ρ}_{1} ⋆ {v}_{sk}^{2}} * \frac{Δ {P}_{sk}}{Δ {L}_{sk}} \qquad ( 2 )$$

The corresponding actual friction coefficient ratios yₖ are shown in the table below, where yₖ=f_{ck}/fₛₖ.

In this embodiment, an average viscosity of the first offline drilling fluid is μ₁=0.00711 Pa·s.

The Dean numbers Dₙₖ of the first offline drilling fluid flowing through the curved pipe for 24 times are calculated according to formula (6) and are shown in the table below.

$${D}_{n k} = \frac{{ρ}_{1} ⋆ {v}_{c k} ⋆ {d}_{tc 1}}{{μ}_{1}} * \sqrt{\frac{{d}_{tc 1}}{{D}_{c 1}}} \qquad ( 6 )$$

| Number | ΔP_{ck}/ | ΔPₛₖ/ | | | | | | |
|---|---|---|---|---|---|---|---|---|
| of times | ΔL_{ck} | ΔLₛₖ | vₛₖ | _{vck} | fₛₖ | f_{ck} | yₖ | Dₙₖ |
| 1 | 0.576 | 0.590 | 0.169 | 0.170 | 0.106 | 0.106 | 1.001 | 29.557 |
| 2 | 0.651 | 0.727 | 0.235 | 0.237 | 0.062 | 0.068 | 1.091 | 50.588 |
| 3 | 0.674 | 0.777 | 0.264 | 0.266 | 0.051 | 0.058 | 1.127 | 61.461 |
| 4 | 0.691 | 0.830 | 0.274 | 0.276 | 0.049 | 0.057 | 1.175 | 64.702 |
| 5 | 0.718 | 0.873 | 0.304 | 0.307 | 0.041 | 0.049 | 1.187 | 76.722 |
| 6 | 0.748 | 0.927 | 0.327 | 0.330 | 0.037 | 0.045 | 1.211 | 85.333 |
| 7 | 0.777 | 0.979 | 0.348 | 0.351 | 0.034 | 0.042 | 1.231 | 92.785 |
| 8 | 0.807 | 1.023 | 0.374 | 0.377 | 0.030 | 0.038 | 1.240 | 103.122 |
| 9 | 0.827 | 1.064 | 0.397 | 0.401 | 0.028 | 0.035 | 1.257 | 113.686 |
| 10 | 0.909 | 1.169 | 0.440 | 0.444 | 0.025 | 0.031 | 1.257 | 126.935 |
| 11 | 0.936 | 1.242 | 0.482 | 0.486 | 0.021 | 0.028 | 1.297 | 147.922 |
| 12 | 0.973 | 1.295 | 0.503 | 0.508 | 0.020 | 0.026 | 1.301 | 155.213 |
| 13 | 0.999 | 1.350 | 0.543 | 0.548 | 0.018 | 0.024 | 1.321 | 176.185 |
| 14 | 1.031 | 1.408 | 0.571 | 0.576 | 0.017 | 0.022 | 1.334 | 188.652 |
| 15 | 1.071 | 1.468 | 0.591 | 0.597 | 0.016 | 0.022 | 1.340 | 194.715 |
| 16 | 1.111 | 1.525 | 0.616 | 0.622 | 0.015 | 0.021 | 1.341 | 203.773 |
| 17 | 1.111 | 1.589 | 0.631 | 0.637 | 0.015 | 0.021 | 1.398 | 213.799 |
| 18 | 1.142 | 1.627 | 0.665 | 0.671 | 0.014 | 0.019 | 1.393 | 230.859 |
| 19 | 1.178 | 1.694 | 0.696 | 0.703 | 0.013 | 0.018 | 1.405 | 245.566 |
| 20 | 1.196 | 1.785 | 0.723 | 0.729 | 0.012 | 0.018 | 1.459 | 260.502 |
| 21 | 1.243 | 1.845 | 0.769 | 0.776 | 0.011 | 0.016 | 1.450 | 283.448 |
| 22 | 1.262 | 1.904 | 0.778 | 0.785 | 0.011 | 0.016 | 1.475 | 286.099 |
| 23 | 1.294 | 1.977 | 0.809 | 0.817 | 0.010 | 0.016 | 1.494 | 301.892 |
| 24 | 1.360 | 2.089 | 0.879 | 0.887 | 0.009 | 0.014 | 1.502 | 338.859 |

Three prediction models are fitted by the actual friction coefficient ratios yk and the Dean numbers Dₙₖ as follows:

a first prediction model:

ŷ₁ₖ=a*Dₙₖ^{b}+c (3)

where, a=0.035966, b=0.5, and c=0.855298.

a second prediction model:

$${\hat{y}}_{2 k} = 1 + \frac{{a}^{*} {D}_{n k}^{b}}{70 + {D}_{n k}} \qquad ( 4 )$$

where, a=0.052896, and b=1.421332.

a third prediction model:

ŷ₃ₖ=1+a*(log₁₀Dₙₖ)^{b} (5)

where, a=0.016495, and b=3.709336.

The predicted friction coefficients of the third prediction models are shown in the table below.

| Number | | | |
|---|---|---|---|
| of times | ŷ₁ₖ | ŷ₂ₖ | ŷ₃ₖ |
| 1 | 1.051 | 1.065 | 1.069 |
| 2 | 1.111 | 1.116 | 1.119 |
| 3 | 1.137 | 1.140 | 1.143 |
| 4 | 1.145 | 1.147 | 1.149 |
| 5 | 1.170 | 1.172 | 1.173 |
| 6 | 1.188 | 1.189 | 1.189 |
| 7 | 1.202 | 1.203 | 1.203 |
| 8 | 1.221 | 1.222 | 1.221 |
| 9 | 1.239 | 1.241 | 1.239 |
| 10 | 1.261 | 1.262 | 1.260 |
| 11 | 1.293 | 1.295 | 1.292 |
| 12 | 1.303 | 1.305 | 1.303 |
| 13 | 1.333 | 1.335 | 1.332 |
| 14 | 1.349 | 1.351 | 1.348 |
| 15 | 1.357 | 1.359 | 1.356 |
| 16 | 1.369 | 1.370 | 1.368 |
| 17 | 1.381 | 1.382 | 1.380 |
| 18 | 1.402 | 1.402 | 1.401 |
| 19 | 1.419 | 1.418 | 1.418 |
| 20 | 1.436 | 1.434 | 1.435 |
| 21 | 1.461 | 1.458 | 1.460 |
| 22 | 1.464 | 1.461 | 1.463 |
| 23 | 1.480 | 1.476 | 1.479 |
| 24 | 1.517 | 1.510 | 1.516 |

A correlation R₁₁² between the actual friction coefficient ratio yₖ and a predicted friction coefficient ratio of the first prediction model, a correlation R₁₂² between the actual friction coefficient ratio yₖ and a predicted friction coefficient ratio of the second prediction model and a correlation R₁₃² between the actual friction coefficient ratio yₖ and a predicted friction coefficient ratio of the third prediction model are calculated according to formulas (7), (8) and (9), respectively.

Through calculation, R₁₁²=0.976421, R₁₂²=0.972209 and R₁₃²=0.971412.

To sum up, in this embodiment, for the first offline drilling fluid, the first prediction model is the optimal prediction model. According to the optimal prediction model, the relationship constants between the friction coefficients are a=0.035966, b=0.5 and c=0.855298, which are used for the on-site measurement on the rheological property of the drilling fluid.

The on-site measurement on the rheological property of the drilling fluid in the curved pipe is described below.

In a first embodiment of the on-site measurement, the density of a first on-site drilling fluid is ρ₂=1,003 kg/m³.

The on-site curved pipe is a helical pipe, which has a total volume V₂=1.04 l and a length len₂=5.57476 m.

An inner diameter of the on-site curved pipe is calculated as d_{tc2}=0.01051 m:

$${d}_{tc 2} = \sqrt{\frac{{4}^{*} {V}_{2}}{{π}^{*} {len}_{2}}} \qquad ( 11 )$$

In this embodiment, a first offline drilling fluid flows through the on-site pipe for i=24 times.

A flow velocity v_{ci} of the first on-site drilling fluid at the i-th time in the curved pipe and an average pressure difference thereof in the curved pipe ΔP_{ci}/ΔL_{ci} are shown in the table below.

The measured parameters of the on-site drilling fluid in the curved pipe are shown in the table below. The flow velocity of the drilling fluid is increased in the curved pipe in an ascending order to keep a laminar flow state of the drilling fluid.

| | Rate | | | | |
|---|---|---|---|---|---|
| Number | of flow | Temperature | | | |
| of times | (lpm) | (° C.) | ΔP_{ci}/ΔL_{ci} | ρ₂ | v_{ci} |
| 1 | 0.8882 | 32.5 | 0.590 | 1003.9 | 0.1705 |
| 2 | 1.2356 | 32.5 | 0.727 | 1002.7 | 0.2372 |
| 3 | 1.3858 | 32.5 | 0.777 | 1002.5 | 0.2660 |
| 4 | 1.4388 | 32.5 | 0.830 | 1003.4 | 0.2762 |
| 5 | 1.5973 | 32.5 | 0.873 | 1004.1 | 0.3066 |
| 6 | 1.7200 | 32.5 | 0.927 | 1003.3 | 0.3302 |
| 7 | 1.8269 | 32.5 | 0.979 | 1004.2 | 0.3507 |
| 8 | 1.9638 | 32.5 | 1.023 | 1002.7 | 0.3770 |
| 9 | 2.0876 | 32.5 | 1.064 | 1002.9 | 0.4007 |
| 10 | 2.3133 | 32.5 | 1.169 | 1002.6 | 0.4441 |
| 11 | 2.5336 | 32.5 | 1.242 | 1002.8 | 0.4864 |
| 12 | 2.6463 | 32.5 | 1.295 | 1002.7 | 0.5080 |
| 13 | 2.8562 | 32.5 | 1.350 | 1002.8 | 0.5483 |
| 14 | 3.0025 | 32.5 | 1.408 | 1003.2 | 0.5764 |
| 15 | 3.1087 | 32.5 | 1.468 | 1002.9 | 0.5968 |
| 16 | 3.2385 | 32.5 | 1.525 | 1003.5 | 0.6217 |
| 17 | 3.3183 | 32.5 | 1.589 | 1002.9 | 0.6370 |
| 18 | 3.4960 | 32.5 | 1.627 | 1002.6 | 0.6711 |
| 19 | 3.6600 | 32.5 | 1.694 | 1003.7 | 0.7026 |
| 20 | 3.7989 | 32.5 | 1.785 | 1003.3 | 0.7293 |
| 21 | 4.0419 | 32.5 | 1.845 | 1002.6 | 0.7759 |
| 22 | 4.0901 | 32.5 | 1.904 | 1002.9 | 0.7852 |
| 23 | 4.2535 | 32.5 | 1.977 | 1003.3 | 0.8165 |
| 24 | 4.6207 | 32.5 | 2.089 | 1003.0 | 0.8870 |

The first on-site drilling fluid flows through the curved pipe for 24 times, and its friction coefficient f_{ci} in the curved pipe is calculated by formula (11), which is shown in the table below.

$${f}_{ci} = \frac{{d}_{tc 2}}{2 {ρ}_{2} ⋆ {v}_{ci}^{2}} * \frac{Δ {P}_{ci}}{Δ {L}_{ci}} \qquad ( 10 )$$

The Reynolds number Rₑᵢ of the on-site curved pipe is calculated according to the selected optimal offline model. In this embodiment, the optimal prediction model is the first prediction model: ŷ₁ₖ=a*Dₙₖ^{b}+c, where the relationship constants between the friction coefficients are respectively: a=0.035966, b=0.5, and c=0.855298.

The Reynolds number Rₑᵢ of the on-site curved pipe is expressed by formula (12):

$${f}_{ci} = \frac{16}{{R}_{ei}} ( {{a}^{*} ( {{R}_{ei}}^{*} \sqrt{\frac{{d}_{tc 2}}{{D}_{c 2}}} )}^{b} + c ) \qquad ( 12 )$$

The Reynolds number Rₑᵢ of the on-site curved pipe is shown in the table below.

According to the Reynolds number Rₑᵢ of the on-site curved pipe, a shear stress τwᵢ of the drilling fluid in the on-site curved pipe is calculated, which is expressed by formula (15), and is shown in the table below.

$${τ}_{wi} = \frac{8 {{ρ}_{2}}^{*} {v}_{ci}^{2}}{{R}_{ei}} \qquad ( 15 )$$

In this embodiment, an intermediate parameter Nᵢ is calculated by a binomial fitting method according to formula (17), which is shown in the table below.

$${N}_{i} = \frac{d ( ln {τ}_{{w}_{i}} )}{d ( ln \frac{{8}^{*} {v}_{ci}}{{d}_{tc 2}} )} \qquad ( 17 )$$

A shear rate γᵢ of the first drilling fluid in the on-site curved pipe is calculated according to formula (16), which is shown in the table below.

$${γ}_{l} = \frac{{8}^{*} {v}_{ci}}{{d}_{tc 2}} * \frac{{3}^{*} {N}_{i} + 1}{{4}^{*} {N}_{i}} \qquad ( 16 )$$

| Number | | | | | |
|---|---|---|---|---|---|
| of times | f_{ci} | R_{ci} | τwᵢ | Nᵢ | γᵢ |
| 1 | 0.11 | 158.92 | 1.47 | 0.47 | 166.99 |
| 2 | 0.07 | 263.11 | 1.72 | 0.50 | 225.78 |
| 3 | 0.06 | 316.51 | 1.79 | 0.51 | 250.91 |
| 4 | 0.06 | 320.14 | 1.91 | 0.51 | 259.74 |
| 5 | 0.05 | 384.57 | 1.96 | 0.53 | 286.06 |
| 6 | 0.05 | 425.27 | 2.06 | 0.53 | 306.33 |
| 7 | 0.04 | 459.71 | 2.15 | 0.54 | 323.95 |
| 8 | 0.04 | 516.12 | 2.21 | 0.55 | 346.44 |
| 9 | 0.04 | 569.37 | 2.26 | 0.55 | 366.69 |
| 10 | 0.03 | 649.48 | 2.44 | 0.56 | 403.50 |
| 11 | 0.03 | 751.29 | 2.53 | 0.57 | 439.27 |
| 12 | 0.03 | 793.06 | 2.61 | 0.58 | 457.51 |
| 13 | 0.02 | 908.07 | 2.66 | 0.58 | 491.39 |
| 14 | 0.02 | 975.29 | 2.73 | 0.59 | 514.94 |
| 15 | 0.02 | 1008.00 | 2.84 | 0.59 | 532.01 |
| 16 | 0.02 | 1064.98 | 2.91 | 0.60 | 552.83 |
| 17 | 0.02 | 1073.93 | 3.03 | 0.60 | 565.62 |
| 18 | 0.02 | 1186.55 | 3.05 | 0.61 | 594.03 |
| 19 | 0.02 | 1267.02 | 3.13 | 0.61 | 620.20 |
| 20 | 0.02 | 1301.98 | 3.28 | 0.61 | 642.33 |
| 21 | 0.02 | 1458.08 | 3.31 | 0.62 | 680.96 |
| 22 | 0.02 | 1444.81 | 3.42 | 0.62 | 688.62 |
| 23 | 0.02 | 1521.06 | 3.52 | 0.63 | 714.54 |
| 24 | 0.01 | 1750.53 | 3.61 | 0.63 | 772.62 |

According to the shear stress τwᵢ and the shear rate γᵢ of the first on-site drilling fluid, at least three on-site models are fitted, which are respectively:

a first on-site model:

\hat{τ}w₁ᵢ=YP+PV*γᵢ (18)

where, PV=0.00354, and YP=0.95267.

a second on-site model:

\hat{τ}w₂ᵢ=K*γᵢⁿ (19),

where K=0.0622, and n=0.6105.

a third on-site model:

\hat{τ}w₂ᵢ=τ₀+K*γᵢⁿ (20),

where, n=0.7991, K=0.0151, and τ₀=0.581.

Then the following parameters are respectively calculated:

a correlation R₂₁² between the shear stress τwᵢ of the first on-site drilling fluid and a predicted shear stress of the first on-site model;

a correlation R₂₂² between the shear stress τwᵢ of the first on-site drilling fluid and a predicted shear stress of the second on-site model; and

a correlation R₂₃² between the shear stress τwᵢ of the first on-site drilling fluid and a predicted shear stress of the third on-site model.

These parameters are calculated according to formulas (21), (22) and (23), respectively.

$$\begin{matrix}{R}_{21}^{2} = 1 - \frac{{\sum }_{i = 1}^{m} {( τ {w}_{i} - \hat{τ {w}_{1 i}} )}^{2}}{{\sum }_{i = 1}^{m} {( τ w - \overline{τ w} )}^{2}} & ( 21 ) \\ {R}_{22}^{2} = 1 - \frac{{\sum }_{i = 1}^{m} {( τ {w}_{i} - \hat{τ {w}_{2 i}} )}^{2}}{{\sum }_{i = 1}^{m} {( τ w - \overline{τ w} )}^{2}} & ( 22 ) \\ {R}_{23}^{2} = 1 - \frac{{\sum }_{i = 1}^{m} {( τ {w}_{i} - \hat{τ {w}_{3 i}} )}^{2}}{{\sum }_{i = 1}^{m} {( τ w - \overline{τ w} )}^{2}} & ( 23 )\end{matrix}$$

Through calculation, R₂₁²=0.9950, R₂₂²=0.9951 and R₂₃²=0.9964. The third on-site model has the maximum correlation and is most in line with the actual situation, so the third on-site model is selected as the final model for calculating other viscosity data.

The on-site measurement results of the third on-site model are compared with those of a 6-speed viscometer (Fann35), which shows that the third on-site model is the most suitable.

| Measurement | Measuring | | | | | | | | |
|---|---|---|---|---|---|---|---|---|---|
| instrument | temperature/° C. | θ600 | θ300 | θ200 | θ100 | θ6 | θ3 | PV | YP |
| Fann35 (control) | 34 | 7 | 5 | 4 | 3 | 1 | 0.5 | 0.002 | 1.533 |
| System measurement | 34 | 8.6 | 5.4 | 4.3 | 2.9 | 1.3 | 1.2 | 0.0032 | 1.1538 |
| device | | | | | | | | | |

If the viscosity data calculated by the first on-site model is directly selected without performing on-site model optimization, as shown in the table below, the deviation will increase significantly. The difference percentage of viscosity corresponding to θ6 is 0.9/1=90%, the difference percentage of viscosity corresponding to θ3 is 1.4/0.5=280%, and the difference percentage of YP is 0.5804/1.533=38%. The calculation of the preferred third model of the present invention shows that the difference percentage of viscosity corresponding to θ6 is 0.3/1=30%, the difference percentage of viscosity corresponding to θ3 is 0.7/0.5=140%, and the difference percentage of YP is 0.3792/1.533=25%.

In conclusion, compared with the measurement results of Fann35, the calculation results of the optimal on-site model (third on-site model) determined by the correlations of the actual shear stress τwᵢ and the predicted shear stresses of the on-site models are the most accurate.

| Measurement | Measuring | | | | | | | | |
|---|---|---|---|---|---|---|---|---|---|
| instrument | temperature/° C. | θ600 | θ300 | θ200 | θ100 | θ6 | θ3 | PV | YP |
| Fann35 (control) | 34 | 7 | 5 | 4 | 3 | 1 | 0.5 | 0.002 | 1.533 |
| System measurement | 34 | 9.0 | 5.4 | 4.2 | 3.0 | 1.9 | 1.9 | 0.0035 | 0.9526 |
| device | | | | | | | | | |

A second embodiment is described below.

In the second embodiment of offline checking, the offline curved pipe has a total volume V₁=1.04 and a length len₁=5.57476 m; the offline straight pipe has an inner diameter dₜₛ₁=0.01056 m; and a second offline drilling fluid has a density ρ₁=1,953 kg/m³.

The inner diameter of the offline curved pipe is calculated as: d_{tc1}=0.01051 m:

$${d}_{tc 1} = \sqrt{\frac{{4}^{*} {V}_{1}}{{π}^{*} {len}_{1}}} \qquad ( 4 )$$

In this embodiment, the second offline drilling fluid flows through the pipe for k=17 times.

A flow velocity vₛₖ of the second offline drilling fluid at the k-th time in a straight pipe and a flow velocity v_{ck} thereof at the k-th time in the curved pipe are shown in the table below.

An average pressure difference ΔP_{ck}/ΔL_{ck} of the offline curved pipe and an average pressure difference ΔPₛₖ/ΔLₛₖ of the offline straight pipe are also shown in the table below.

The second offline drilling fluid flows through for 17 times, and its friction coefficient f_{ck} in the offline curved pipe and friction coefficient fₛₖ in the offline straight pipe are calculated by formulas (1) and (2), and are shown in the table below.

$$\begin{matrix}{f}_{ck} = \frac{{d}_{tc 1}}{2 {{ρ}_{1}}^{*} {v}_{ck}^{2}} * \frac{Δ {P}_{ck}}{Δ L} & ( 1 ) \\ {f}_{sk} = \frac{{d}_{ts 1}}{2 {{ρ}_{1}}^{*} {v}_{sk}^{2}} * \frac{Δ {P}_{sk}}{Δ L} & ( 2 )\end{matrix}$$

The corresponding actual friction coefficient ratios yₖ are shown in the table below, where yₖ=f_{ck}/fₛₖ.

In this embodiment, an average viscosity of the second offline drilling fluid is μ₁=0.02639 Pa·s.

The Dean numbers Dₙₖ of the second offline drilling fluid flowing through the curved pipe for 17 times are calculated according to formula (6), which are shown in the table below.

$${D}_{nk} = \frac{{{ρ}_{1}}^{*} {{v}_{ck}}^{*} {d}_{tc 1}}{{μ}_{1}} * \sqrt{\frac{{d}_{tc 1}}{{D}_{c 1}}} \qquad ( 6 )$$

| Number | ΔP_{ck}/ | ΔPₛₖ/ | | | | | | |
|---|---|---|---|---|---|---|---|---|
| of times | ΔL_{ck} | ΔLₛₖ | vₛₖ | v_{ck} | fₛₖ | f_{ck} | yₖ | Dₙₖ |
| 1 | 6.249 | 8.759 | 0.80 | 0.81 | 0.03 | 0.04 | 1.37 | 119.74 |
| 2 | 7.020 | 9.999 | 0.91 | 0.91 | 0.02 | 0.03 | 1.39 | 135.57 |
| 3 | 7.447 | 10.907 | 0.97 | 0.98 | 0.02 | 0.03 | 1.43 | 145.85 |
| 4 | 8.008 | 12.182 | 1.07 | 1.08 | 0.02 | 0.03 | 1.49 | 166.80 |
| 5 | 8.420 | 12.888 | 1.11 | 1.12 | 0.02 | 0.03 | 1.50 | 167.88 |
| 6 | 8.583 | 13.091 | 1.13 | 1.14 | 0.02 | 0.03 | 1.49 | 172.00 |
| 7 | 9.179 | 14.505 | 1.21 | 1.22 | 0.02 | 0.03 | 1.54 | 184.56 |
| 8 | 9.603 | 15.250 | 1.29 | 1.30 | 0.02 | 0.02 | 1.55 | 199.90 |
| 9 | 10.128 | 16.783 | 1.37 | 1.38 | 0.01 | 0.02 | 1.62 | 215.73 |
| 10 | 10.338 | 17.224 | 1.41 | 1.43 | 0.01 | 0.02 | 1.63 | 224.42 |
| 11 | 10.880 | 18.257 | 1.47 | 1.48 | 0.01 | 0.02 | 1.64 | 230.18 |
| 12 | 13.825 | 24.076 | 1.82 | 1.83 | 0.01 | 0.02 | 1.70 | 277.64 |
| 13 | 13.706 | 24.131 | 1.83 | 1.85 | 0.01 | 0.02 | 1.72 | 284.93 |
| 14 | 14.606 | 26.401 | 1.96 | 1.98 | 0.01 | 0.02 | 1.77 | 305.94 |
| 15 | 15.228 | 27.892 | 2.07 | 2.09 | 0.01 | 0.02 | 1.79 | 326.25 |
| 16 | 16.025 | 29.395 | 2.14 | 2.16 | 0.01 | 0.02 | 1.79 | 333.56 |
| 17 | 16.940 | 30.332 | 2.21 | 2.23 | 0.01 | 0.02 | 1.75 | 333.69 |

Three prediction models are fitted by the actual friction coefficient ratios yk and the Dean numbers Dₙₖ as follows:

a first prediction model:

ŷ₁ₖ=a*Dₙₖ^{b}+c (3)

where, a=0.0576, b=0.5, and c=0.745.

a second prediction model:

$${\hat{y}}_{2 k} = 1 + \frac{{a}^{*} {D}_{nk}^{b}}{70 + {D}_{nk}} \qquad ( 4 )$$

where, a=0.0644, and b=1.4654.

a third prediction model:

ŷ₃ₖ=1+a*(log₁₀Dₙₖ)^{b} (5)

where, a=0.0231, and b=3.8241.

The predicted friction coefficients of the third prediction models are shown in the table below.

| Number | | | |
|---|---|---|---|
| of times | ŷ₁ₖ | ŷ₂ₖ | ŷ₃ₖ |
| 1 | 1.376 | 1.377 | 1.379 |
| 2 | 1.416 | 1.417 | 1.418 |
| 3 | 1.441 | 1.442 | 1.443 |
| 4 | 1.489 | 1.491 | 1.490 |
| 5 | 1.492 | 1.493 | 1.492 |
| 6 | 1.501 | 1.502 | 1.501 |
| 7 | 1.528 | 1.529 | 1.528 |
| 8 | 1.560 | 1.561 | 1.560 |
| 9 | 1.591 | 1.593 | 1.591 |
| 10 | 1.608 | 1.610 | 1.608 |
| 11 | 1.619 | 1.620 | 1.619 |
| 12 | 1.705 | 1.705 | 1.704 |
| 13 | 1.718 | 1.717 | 1.717 |
| 14 | 1.753 | 1.752 | 1.752 |
| 15 | 1.786 | 1.784 | 1.785 |
| 16 | 1.797 | 1.795 | 1.796 |
| 17 | 1.798 | 1.795 | 1.797 |

A correlation R₁₁² between the actual friction coefficient ratio yₖ and a predicted friction coefficient ratio of the first prediction model, a correlation R₁₂² between the actual friction coefficient ratio yₖ and a predicted friction coefficient ratio of the second prediction model and a correlation R₁₃² between the actual friction coefficient ratio yₖ and a predicted friction coefficient ratio of the third prediction model are calculated according to formulas (7), (8) and (9), respectively.

Through calculation, R₁₁²=0.9833, R₁₂²=0.9843 and R₁₃²=0.9828.

To sum up, in this embodiment, for the second offline drilling fluid, the first prediction model is the optimal prediction model. According to the optimal prediction model, the relationship constants between the friction coefficients are a=0.0644 and b=1.4654, which are used for the on-site measurement on the rheological property of the drilling fluid.

The on-site measurement on the rheological property of the drilling fluid in the curved pipe is described below.

In a second embodiment of the on-site measurement, the density of a second on-site drilling fluid is ρ₂=1,300 kg/m³.

The on-site curved pipe has a total volume V₂=1.04 l and a length len₂=5.57476 m.

An inner diameter of the on-site curved pipe is calculated as d_{tc2}=0.01051 m:

$${d}_{tc 2} = \sqrt{\frac{{4}^{*} {∇}_{2}}{{π}^{*} {len}_{2}}} \qquad ( 11 )$$

In this embodiment, a second offline drilling fluid flows through the on-site pipe for i=17 times.

A i-th flow velocity v_{ci} of the second on-site drilling fluid at the i-th time in the curved pipe and an average pressure difference thereof in the curved pipe ΔP_{ci}/ΔL_{ci} are shown in the table below.

The measured parameters of the on-site drilling fluid in the curved pipe are shown in the table below. The flow velocity of the drilling fluid is increased in the curved pipe in an ascending order to keep a laminar flow state of the drilling fluid.

| | Rate | | | | |
|---|---|---|---|---|---|
| Number | of flow | Temperature | | | |
| of times | (lpm) | (° C.) | ΔPci/ΔLci | ρ2 | vci |
| 1 | 4.212 | 29 | 8.759 | 1960.5 | 0.809 |
| 2 | 4.761 | 29 | 9.999 | 1951.0 | 0.914 |
| 3 | 5.088 | 29 | 10.907 | 1950.2 | 0.977 |
| 4 | 5.642 | 29 | 12.182 | 1950.2 | 1.083 |
| 5 | 5.813 | 29 | 12.888 | 1944.5 | 1.116 |
| 6 | 5.933 | 29 | 13.091 | 1949.2 | 1.139 |
| 7 | 6.358 | 29 | 14.505 | 1947.9 | 1.221 |
| 8 | 6.776 | 29 | 15.250 | 1943.1 | 1.301 |
| 9 | 7.207 | 29 | 16.783 | 1955.0 | 1.384 |
| 10 | 7.425 | 29 | 17.224 | 1956.0 | 1.425 |
| 11 | 7.716 | 29 | 18.257 | 1955.1 | 1.481 |
| 12 | 9.544 | 29 | 24.076 | 1958.6 | 1.832 |
| 13 | 9.631 | 29 | 24.131 | 1956.9 | 1.849 |
| 14 | 10.306 | 29 | 26.401 | 1955.3 | 1.978 |
| 15 | 10.868 | 29 | 27.892 | 1955.2 | 2.086 |
| 16 | 11.263 | 29 | 29.395 | 1958.4 | 2.162 |
| 17 | 11.595 | 29 | 30.332 | 1954.3 | 2.226 |

The second on-site drilling fluid flows through the curved pipe for 17 times, and its friction coefficient f_{ci} in the curved pipe is calculated by formula (11), which is shown in the table below.

$${f}_{ci} = \frac{{d}_{tc 2}}{2 {{ρ}_{2}}^{*} {v}_{ci}^{2}} * \frac{Δ {P}_{ci}}{Δ {L}_{ci}} \qquad ( 10 )$$

The Reynolds number Rₑᵢ of the on-site curved pipe is calculated according to the selected optimal offline model. In this embodiment, the optimal prediction model is the second prediction model:

$${\hat{y}}_{2 k} = 1 + \frac{{a}^{*} {D}_{nk}^{b}}{70 + {D}_{nk}} ,$$

where the relationship constants between the friction coefficients are respectively: a=0.0644 and b=1.4654.

The Reynolds number Rₑᵢ of the on-site curved pipe is expressed by formula (12):

$${f}_{ci} = \frac{16}{{R}_{ei}} ( {{a}^{*} ( {{R}_{ei}}^{*} \sqrt{\frac{{d}_{tc 2}}{{D}_{c 2}}} )}^{b} + c ) \qquad ( 12 )$$

The Reynolds number Rₑᵢ of the on-site curved pipe is shown in the table below.

According to the Reynolds number Rₑᵢ of the on-site curved pipe, a shear stress τwᵢ of the drilling fluid in the on-site curved pipe is calculated, which is expressed by formula (15), and is shown in the table below.

$${τ}_{wi} = \frac{8 {{ρ}_{2}}^{*} {v}_{ci}^{2}}{{R}_{ei}} \qquad ( 15 )$$

An intermediate parameter Nᵢ is calculated according to formula (17), which is shown in the table below.

$${N}_{i} = \frac{d ( ln {τ}_{{w}_{i}} )}{d ( ln \frac{{8}^{*} {v}_{ci}}{{d}_{tc 2}} )} \qquad ( 17 )$$

A shear rate γᵢ of the second drilling fluid in the on-site curved pipe is calculated according to formula (16), which is shown in the table below.

$${γ}_{i} = \frac{{8}^{*} {v}_{ci}}{{d}_{t c 2}} * \frac{{3}^{*} {N}_{i} + 1}{{4}^{*} {N}_{i}} \qquad ( 16 )$$

| Number | | | | | |
|---|---|---|---|---|---|
| of times | f_{ci} | Rₑᵢ | τwᵢ | Nᵢ | γᵢ |
| 1 | 0.0359 | 614.0792 | 16.6964 | 0.9448 | 624.1797 |
| 2 | 0.0322 | 707.0957 | 18.4418 | 0.9517 | 704.3091 |
| 3 | 0.0308 | 750.4231 | 19.8311 | 0.9554 | 751.8029 |
| 4 | 0.0280 | 852.8917 | 21.4602 | 0.9612 | 832.4728 |
| 5 | 0.0280 | 853.1906 | 22.7020 | 0.9628 | 857.2334 |
| 6 | 0.0272 | 885.4715 | 22.8450 | 0.9640 | 874.7194 |
| 7 | 0.0263 | 928.1204 | 25.0111 | 0.9678 | 936.3869 |
| 8 | 0.0244 | 1026.7406 | 25.6190 | 0.9714 | 997.0713 |
| 9 | 0.0236 | 1074.6661 | 27.8581 | 0.9748 | 1059.5306 |
| 10 | 0.0228 | 1125.6006 | 28.2435 | 0.9765 | 1091.0656 |
| 11 | 0.0224 | 1153.9089 | 29.7386 | 0.9786 | 1133.1933 |
| 12 | 0.0193 | 1418.3999 | 37.0815 | 0.9905 | 1397.4120 |
| 13 | 0.0190 | 1448.0218 | 36.9546 | 0.9910 | 1409.9395 |
| 14 | 0.0181 | 1540.8543 | 39.7384 | 0.9948 | 1507.3717 |
| 15 | 0.0172 | 1654.1138 | 41.1553 | 0.9978 | 1588.2620 |
| 16 | 0.0169 | 1702.5492 | 43.0205 | 0.9998 | 1645.2677 |
| 17 | 0.0165 | 1761.7294 | 43.9646 | 1.0014 | 1692.9891 |

According to the shear stress τwᵢ and the shear rate γᵢ of the second on-site drilling fluid, at least three on-site models are fitted, which are respectively:

a first on-site model:

\hat{τ}w₁ᵢ=YP+PV*γᵢ (18)

where, PV=0.026, and YP=0.241.

a second on-site model:

\hat{τ}w₂ᵢ=K*γᵢⁿ (19),

where K=0.0278, and n=0.9917.

a third on-site model:

\hat{τ}w₂ᵢ=τ₀+K*γᵢⁿ (20),

Where, n=0.9991, K=0.0262, and τ₀=0.2146.

Then the following parameters are respectively calculated:

a correlation R₂₁² between the shear stress τwᵢ of the second on-site drilling fluid and a predicted shear stress of the first on-site model;

a correlation R₂₂² between the shear stress τwᵢ of the second on-site drilling fluid and a predicted shear stress of the second on-site model; and

a correlation R₂₃² between the shear stress τwᵢ of the second on-site drilling fluid and a predicted shear stress of the third on-site model.

These parameters are calculated according to formulas (21), (22) and (23), respectively.

$${R}_{21}^{2} = 1 - \frac{{\sum }_{i = 1}^{m} {( {τw}_{i} - τ {\hat{w}}_{1 i} )}^{2}}{{\sum }_{i = 1}^{m} {( {τw}_{i} - \overline{τw} )}^{2}} \qquad ( 21 )$$

$${R}_{22}^{2} = 1 - \frac{{\sum }_{i = 1}^{m} {( {τw}_{i} - τ {\hat{w}}_{2 i} )}^{2}}{{\sum }_{i = 1}^{m} {( {τw}_{i} - \overline{τw} )}^{2}} \qquad ( 22 )$$

$${R}_{23}^{2} = 1 - \frac{{\sum }_{i = 1}^{m} {( {τw}_{i} - τ {\hat{w}}_{3 i} )}^{2}}{{\sum }_{i = 1}^{m} {( {τw}_{i} - \overline{τw} )}^{2}} \qquad ( 23 )$$

Through calculation, R₂₁²=0.998873, R₂₂²=0.996445 and R₂₃²=0.998873. The first and third on-site models have the maximum correlation and are most in line with the actual situation, so the first and third on-site models are selected as the final models for calculating other viscosity data.

The on-site measurement results of the first on-site model are compared with those of the 6-speed viscometer, which shows that the first on-site model is the most suitable.

| Measurement | Measuring | | | | | | |
|---|---|---|---|---|---|---|---|
| instrument | temperature/° C. | θ600 | θ300 | θ200 | θ100 | θ6 | θ3 |
| Fann35 (control) | 29 | 50 | 27 | 19 | 11 | 1 | 0.5 |
| System measurement | 29 | 52.42 | 26.45 | 17.79 | 9.13 | 0.99 | 0.73 |
| device | | | | | | | |

The on-site measurement results of the third on-site model are compared with those of the 6-speed viscometer, which shows that the third on-site model is the most suitable.

| Measurement | Measuring | | | | | | | | | |
|---|---|---|---|---|---|---|---|---|---|---|
| instrument | temperature/° C. | θ600 | θ300 | θ200 | θ100 | θ6 | θ3 | n | K | τ0 |
| Fann35 (control) | 29 | 50 | 27 | 19 | 11 | 1 | 0.5 | 0.9015 | 0.0490 | 0.26 |
| System measurement | 29 | 52.42 | 26.44 | 17.77 | 9.10 | 0.94 | 0.68 | 0.9991 | 0.0262 | 0.21 |
| device | | | | | | | | | | |

If the viscosity data calculated by the second on-site model is directly selected without performing on-site model optimization, as shown in the table below, the deviation will increase significantly. The difference percentage of viscosity corresponding to θ100 is 2.13/11=19% and the difference percentage of viscosity corresponding to θ6 is 0.46/1=46%. The calculation of the preferred third model of the present invention shows that the difference percentage of viscosity corresponding to θ100 is 1.9/11=17% and the difference percentage of viscosity corresponding to θ6 is 0.06/1=6%. The calculation of the preferred first model of the present invention shows that the difference percentage of viscosity corresponding to θ100 is 1.83/11=17% and the difference percentage of viscosity corresponding to θ6 is 0.01/1=1%.

| | |
|---|---|
| Measurement | Measuring |

| instrument | temperature/° C. | θ600 | θ300 | θ200 | θ100 | θ6 | θ3 |
|---|---|---|---|---|---|---|---|
| Fann35 (control) | 29 | 50 | 27 | 19 | 11 | 1 | 0.5 |
| System | | | | | | | |
| measurement | 29 | 52.43 | 26.37 | 17.64 | 8.87 | 0.54 | 0.27 |
| device | | | | | | | |

In conclusion, compared with the measurement results of Fann35, the calculation results of the optimal on-site models (the first and third on-site models) determined by the correlations of the actual shear stress τwᵢ and the predicted shear stresses of the on-site models are the most accurate.

A third embodiment is described below.

In the third embodiment of offline checking, the offline curved pipe has a total volume V₁=1.04 l and a length len₁=5.57476 m; the offline straight pipe has an inner diameter dₜₛ₁=0.01056 m; and a third offline drilling fluid has a density ρ₁=1,227 kg/m³.

The inner diameter of the offline curved pipe is calculated as: d_{tc1}=0.01051 m:

$${d}_{tc 1} = \sqrt{\frac{{4}^{*} {V}_{1}}{{π}^{*} {len}_{1}}} \qquad ( 4 )$$

In this embodiment, the third offline drilling fluid flows through the pipe for k=13 times.

A flow velocity vₛₖ of the third offline drilling fluid at the k-th time in a straight pipe and a flow velocity v_{ck} thereof at the k-th time in the curved pipe are shown in the table below.

An average pressure difference ΔP_{ck}/ΔL_{ck} of the offline curved pipe and an average pressure difference ΔPₛₖ/ΔLₛₖ of the offline straight pipe are also shown in the table below.

The third offline drilling fluid flows through the pipe for 13 times, and its friction coefficient f_{ck} in the curved pipe and friction coefficient fₛₖ in the straight pipe are calculated by formulas (1) and (2), and are shown in the table below.

$${f}_{c k} = \frac{{d}_{tc 1}}{2 {ρ}_{1} ⋆ {v}_{c k}^{2}} * \frac{Δ {P}_{c k}}{Δ {L}_{c k}} \qquad ( 1 )$$

$${f}_{s k} = \frac{{d}_{ts 1}}{2 {ρ}_{1} ⋆ {v}_{sk}^{2}} * \frac{Δ {P}_{sk}}{Δ {L}_{sk}} \qquad ( 2 )$$

The corresponding actual friction coefficient ratios yₖ are shown in the table below, where yₖ=f_{ck}/fₛₖ.

In this embodiment, an average viscosity of the third offline drilling fluid is μ₁=0.01455 Pa·s.

The Dean numbers Dₙₖ of the third offline drilling fluid flowing through the curved pipe for 13 times are calculated according to formula (6), which are shown in the table below.

$${D}_{n k} = \frac{{ρ}_{1} ⋆ {v}_{c k} ⋆ {d}_{tc 1}}{{μ}_{1}} * \sqrt{\frac{{d}_{tc 1}}{{D}_{c 1}}} \qquad ( 6 )$$

| Number | ΔP_{ck}/ | ΔPₛₖ/ | | | | | | |
|---|---|---|---|---|---|---|---|---|
| of times | ΔL_{ck} | ΔLₛₖ | vₛₖ | _{vck} | fₛₖ | f_{ck} | yₖ | Dₙₖ |
| 1 | 2.634 | 3.108 | 0.56 | 0.56 | 0.04 | 0.04 | 1.15 | 85.88 |
| 2 | 2.788 | 3.381 | 0.61 | 0.61 | 0.03 | 0.04 | 1.19 | 96.66 |
| 3 | 2.881 | 3.555 | 0.64 | 0.65 | 0.03 | 0.04 | 1.21 | 105.05 |
| 4 | 3.050 | 3.808 | 0.69 | 0.70 | 0.03 | 0.03 | 1.22 | 114.64 |
| 5 | 3.363 | 4.360 | 0.79 | 0.80 | 0.02 | 0.03 | 1.27 | 134.93 |
| 6 | 3.447 | 4.622 | 0.83 | 0.84 | 0.02 | 0.03 | 1.31 | 145.22 |
| 7 | 3.555 | 4.894 | 0.87 | 0.88 | 0.02 | 0.03 | 1.35 | 154.57 |
| 8 | 3.664 | 5.160 | 0.91 | 0.92 | 0.02 | 0.03 | 1.38 | 166.17 |
| 9 | 3.784 | 5.453 | 0.96 | 0.97 | 0.02 | 0.03 | 1.41 | 176.43 |
| 10 | 3.909 | 5.734 | 1.01 | 1.02 | 0.02 | 0.02 | 1.43 | 190.62 |
| 11 | 4.036 | 6.001 | 1.05 | 1.06 | 0.02 | 0.02 | 1.45 | 198.90 |
| 12 | 4.177 | 6.287 | 1.09 | 1.10 | 0.02 | 0.02 | 1.47 | 208.59 |
| 13 | 4.215 | 6.420 | 1.11 | 1.12 | 0.01 | 0.02 | 1.49 | 211.66 |

Three prediction models are fitted by the actual friction coefficient ratios yₖ and the Dean numbers Dₙₖ as follows:

a first prediction model:

ŷ₁ₖ=a*Dₙₖ^{b}+c (3)

where, a=0.0645, b=0.5, and c=0.5424.

a second prediction model:

$${\hat{y}}_{2 k} = 1 + \frac{a ⋆ {D}_{n k}^{b}}{70 + {D}_{n k}} \qquad ( 4 )$$

where, a=0.0045, and b=1.9298.

a third prediction model:

ŷ₃ₖ=1+a*(log₁₀Dₙₖ)^{b} (5)

where, a=0.0025, and b=6.2539.

The predicted friction coefficients of the third prediction models are shown in the table below.

| Number | | | |
|---|---|---|---|
| of times | ŷ₁ₖ | ŷ₂ₖ | ŷ₃ₖ |
| 1 | 1.1401 | 1.1555 | 1.1547 |
| 2 | 1.1765 | 1.1827 | 1.1822 |
| 3 | 1.2035 | 1.2042 | 1.2040 |
| 4 | 1.2330 | 1.2292 | 1.2291 |
| 5 | 1.2916 | 1.2828 | 1.2830 |
| 6 | 1.3196 | 1.3103 | 1.3106 |
| 7 | 1.3443 | 1.3354 | 1.3358 |
| 8 | 1.3738 | 1.3668 | 1.3671 |
| 9 | 1.3991 | 1.3946 | 1.3948 |
| 10 | 1.4329 | 1.4332 | 1.4333 |
| 11 | 1.4520 | 1.4557 | 1.4557 |
| 12 | 1.4739 | 1.4822 | 1.4819 |
| 13 | 1.4807 | 1.4905 | 1.4902 |

A correlation R₁₁² between the actual friction coefficient ratio yₖ and a predicted friction coefficient ratio of the first prediction model, a correlation R₁₂² between the actual friction coefficient ratio yₖ and a predicted friction coefficient ratio of the second prediction model and a correlation R₁₃² between the actual friction coefficient ratio yₖ and a predicted friction coefficient ratio of the third prediction model are calculated according to formulas (7), (8) and (9), respectively.

Through calculation, R₁₁²=0.9923, R₁₂²=0.9948 and R₁₃²=0.9949.

To sum up, in this embodiment, for the third offline drilling fluid, the third prediction model is the optimal prediction model. According to the optimal prediction model, the relationship constants between the friction coefficients are a=0.0025 and b=6.2539, which are used for the on-site measurement on the rheological property of the drilling fluid.

The on-site measurement on the rheological property of the drilling fluid in the curved pipe is described below.

In the third embodiment of the on-site measurement, the density of a third on-site drilling fluid is ρ₃=1,227 kg/m³.

The on-site curved pipe has a total volume V₂=1.04 l and a length len₂=5.57476 m.

An inner diameter of the on-site curved pipe is calculated as d_{tc2}=0.01051 m:

$${d}_{t c 2} = \sqrt{\frac{{4}^{*} {V}_{2}}{{π}^{*} le {n}_{2}}} \qquad ( 11 )$$

In this embodiment, the third offline drilling fluid flows through the on-site pipe for i=13 times.

A i-th flow velocity v_{ci} of the second on-site drilling fluid at the i-th time in the curved pipe and an average pressure difference thereof in the curved pipe ΔP_{ci}/ΔL_{ci} are shown in the table below.

The measured parameters of the on-site drilling fluid in the curved pipe are shown in the table below. The flow velocity of the drilling fluid is increased in the curved pipe in an ascending order to keep a laminar flow state of the drilling fluid.

| | Rate | | | | |
|---|---|---|---|---|---|
| Number | of flow | Temperature | | | |
| of times | (lpm) | (° C.) | ΔP_{ci}/ΔL_{ci} | ρ₂ | vcᵢ |
| 1 | 2.926 | 29 | 3.11 | 1227.7 | 0.5617 |
| 2 | 3.195 | 29 | 3.38 | 1227.5 | 0.6133 |
| 3 | 3.385 | 29 | 3.55 | 1228.0 | 0.6497 |
| 4 | 3.640 | 29 | 3.81 | 1226.4 | 0.6988 |
| 5 | 4.146 | 29 | 4.36 | 1227.3 | 0.7958 |
| 6 | 4.355 | 29 | 4.62 | 1226.6 | 0.8360 |
| 7 | 4.563 | 29 | 4.89 | 1226.8 | 0.8759 |
| 8 | 4.802 | 29 | 5.16 | 1227.3 | 0.9218 |
| 9 | 5.028 | 29 | 5.45 | 1227.5 | 0.9652 |
| 10 | 5.313 | 29 | 5.73 | 1226.9 | 1.0199 |
| 11 | 5.512 | 29 | 6.00 | 1227.9 | 1.0582 |
| 12 | 5.741 | 29 | 6.29 | 1228.7 | 1.1021 |
| 13 | 5.815 | 29 | 6.42 | 1226.2 | 1.1163 |

The second on-site drilling fluid flows through the curved pipe for 13 times, and its friction coefficient f_{ci} in the curved pipe is calculated by formula (11), which is shown in the table below.

$${f}_{ci} = \frac{{d}_{t c 2}}{2 {ρ}_{2} ⋆ {v}_{ci}^{2}} * \frac{Δ {P}_{ci}}{Δ {L}_{ci}} \qquad ( 10 )$$

The Reynolds number Rₑᵢ of the on-site curved pipe is calculated according to the selected optimal offline model. In this embodiment, the optimal prediction model is the third prediction model: ŷ₃ₖ=1+a*(log₁₀ Dₙₖ)^{b}, where the relationship constants between the friction coefficients are respectively: a=0.0025 and b=6.2539.

The Reynolds number Rₑᵢ of the on-site curved pipe is expressed by formula (12):

$${f}_{c i} = \frac{16}{{R}_{e i}} ( 1 + a * {( {log}_{10} ( {R}_{e i} * \sqrt{\frac{{d}_{t c 2}}{{D}_{c 2}}} ) )}^{b} ) \qquad ( 12 )$$

The Reynolds number Rₑᵢ of the on-site curved pipe is shown in the table below.

According to the Reynolds number Rₑᵢ of the on-site curved pipe, a shear stress τwᵢ of the drilling fluid in the on-site curved pipe is calculated, which is expressed by formula (15), and is shown in the table below.

$${τ}_{w i} = \frac{8 {ρ}_{2} ⋆ {v}_{ci}^{2}}{{R}_{e i}} \qquad ( 15 )$$

An intermediate parameter Nᵢ is calculated according to formula (17), which is shown in the table below.

$${N}_{i} = \frac{d ( ln {τ}_{wi} )}{d ( ln \frac{{8}^{*} {v}_{ci}}{{d}_{tc 2}} )} \qquad ( 17 )$$

A shear rate γᵢ of the third drilling fluid in the on-site curved pipe is calculated according to formula (16), which is shown in the table below.

$${γ}_{i} = \frac{{8}^{*} {v}_{ci}}{{d}_{t c 2}} * \frac{{3}^{*} {N}_{i} + 1}{{4}^{*} {N}_{i}} \qquad ( 16 )$$

| Number | | | | |
|---|---|---|---|---|
| of times | f_{ci} | Rₑᵢ | τwᵢ | γᵢ |
| 1 | 0.042 | 438.213 | 7.072 | 474.696 |
| 2 | 0.038 | 491.082 | 7.521 | 518.882 |
| 3 | 0.036 | 534.109 | 7.764 | 550.185 |
| 4 | 0.033 | 589.713 | 8.124 | 592.341 |
| 5 | 0.029 | 699.632 | 8.888 | 675.882 |
| 6 | 0.028 | 739.707 | 9.271 | 710.482 |
| 7 | 0.027 | 779.393 | 9.661 | 744.922 |
| 8 | 0.026 | 838.195 | 9.953 | 784.520 |
| 9 | 0.025 | 885.939 | 10.327 | 822.071 |
| 10 | 0.024 | 970.584 | 10.518 | 869.288 |
| 11 | 0.023 | 1016.091 | 10.825 | 902.452 |
| 12 | 0.022 | 1075.315 | 11.102 | 940.432 |
| 13 | 0.022 | 1080.252 | 11.316 | 952.785 |

According to the shear stress τwᵢ and the shear rate γᵢ of the third on-site drilling fluid, at least three on-site models are fitted, which are respectively:

a first on-site model:

\hat{τ}w₁ᵢ=YP+PV*γᵢ (18)

where, PV=0.0088, and YP=2.98.

a second on-site model:

\hat{τ}w₂ᵢ=K*γᵢⁿ (19),

where, K=0.6715, and n=0.1126.

a third on-site model:

\hat{τ}w₂ᵢ=τ₀+K*γᵢⁿ (20),

where, n=0.6716, K=0.1126, and τ₀=0.001.

Then the following parameters are respectively calculated.

a correlation R₂₁² between the shear stress τwᵢ of the second on-site drilling fluid and a predicted shear stress of the first on-site model;

a correlation R₂₂² between the shear stress τwᵢ of the second on-site drilling fluid and a predicted shear stress of the second on-site model; and

a correlation R₂₃² between the shear stress τwᵢ of the second on-site drilling fluid and a predicted shear stress of the third on-site model.

These parameters are calculated according to formulas (21), (22) and (23), respectively.

$${R}_{21}^{2} = 1 - \frac{{\sum }_{i = 1}^{m} {( {τw}_{i} - τ {\hat{w}}_{1 i} )}^{2}}{{\sum }_{i = 1}^{m} {( {τw}_{i} - \overline{τw} )}^{2}} \qquad ( 21 )$$

$${R}_{22}^{2} = 1 - \frac{{\sum }_{i = 1}^{m} {( {τw}_{i} - τ {\hat{w}}_{2 i} )}^{2}}{{\sum }_{i = 1}^{m} {( {τw}_{i} - \overline{τw} )}^{2}} \qquad ( 22 )$$

$${R}_{23}^{2} = 1 - \frac{{\sum }_{i = 1}^{m} {( {τw}_{i} - τ {\hat{w}}_{3 i} )}^{2}}{{\sum }_{i = 1}^{m} {( {τw}_{i} - \overline{τw} )}^{2}} \qquad ( 23 )$$

Through calculation, R₂₁²=0.996314, R₂₂²=0.997775 and R₂₃²=0.997775. The second and third on-site models have the maximum correlation and are most in line with the actual situation, so the second and third on-site models are selected as the final models for calculating other viscosity data.

The on-site measurement results of the second on-site model are compared with those of the 6-speed viscometer, which shows that the third on-site model is the most suitable.

| Measurement | Measuring | | | | | | | | |
|---|---|---|---|---|---|---|---|---|---|
| instrument | temperature/° C. | θ600 | θ300 | θ200 | θ100 | θ6 | θ3 | n | K |
| Fann35 (control) | 29 | 22 | 14 | 10 | 6.5 | 1 | 0.8 | 0.6521 | 0.1226 |
| System measurement | 29 | 23.1 | 14.5 | 11.1 | 6.9 | 1.0 | 0.7 | 0.6715 | 0.1126 |
| device | | | | | | | | | |

The on-site measurement results of the third on-site model are compared with those of the 6-speed viscometer, which shows that the third on-site model is the most suitable.

| Measurement | Measuring | | | | | | | | | |
|---|---|---|---|---|---|---|---|---|---|---|
| instrument | temperature/° C. | θ600 | θ300 | θ200 | θ100 | θ6 | θ3 | n | K | τ0 |
| Fann35 (control) | 29 | 22 | 14 | 10 | 6.5 | 1 | 0.8 | 0.6835 | 0.0950 | 0.4088 |
| System measurement | 29 | 23.1 | 14.5 | 11.1 | 6.9 | 1.0 | 0.7 | 0.6716 | 0.1126 | 0.001 |
| device | | | | | | | | | | |

If the viscosity data calculated by the first on-site model is directly selected without performing on-site model optimization, as shown in the table below, the deviation will increase significantly. The difference percentage of viscosity corresponding to θ100 is 2.2/6.5=35%, the difference percentage of viscosity corresponding to θ6 is 5/1=500%, and the difference percentage of viscosity corresponding to θ3 is 5.1/0.8=639%. The calculation results of the preferred second and third models of the present invention show that the difference percentage of viscosity corresponding to θ100 is 0.4/6.5=7%, the difference percentage of viscosity corresponding to θ6 is 0.1/5=5% and the difference percentage of viscosity corresponding to θ3 is −0.1/0.8=−17%.

| Measurement | Measuring | | | | | | |
|---|---|---|---|---|---|---|---|
| instrument | temperature/° C. | θ600 | θ300 | θ200 | θ100 | θ6 | θ3 |
| Fann35 | 29 | 22 | 14 | 10 | 6.5 | 1 | 0.8 |
| (control) | | | | | | | |
| System | 29 | 23.4 | 14.6 | 11.7 | 8.7 | 6.0 | 5.9 |
| measurement | | | | | | | |
| device | | | | | | | |

In conclusion, compared with the measurement results of Fann35, the calculation results of the optimal on-site models (the second and third on-site models) determined by the correlations of the actual shear stress τwᵢ and the predicted shear stresses of the on-site models are the most accurate.

The above described are merely preferred embodiments of the present invention, which are not intended to limit the present invention. Any modifications, equivalent replacements and improvements made within the spirit and principle of the present invention should be included in the protection scope of the present invention.

What is claimed is:

1. A method for measuring a rheological property of a drilling fluid by using a curved pipe on site, comprising the following steps:
step 1: deriving relationship constants between friction coefficients of a drilling fluid through offline checking;
step 2: calculating a Reynolds number Rₑᵢ of the drilling fluid in an on-site curved pipe according to the relationship constants between the friction coefficients of the drilling fluid and a friction coefficient f_{ci} of the drilling fluid in the on-site curved pipe, wherein i denotes a number of times the drilling fluid flows through the on-site curved pipe, and i is a positive integer not less than 2;
step 3: calculating an actual shear stress τwᵢ of the drilling fluid in the on-site curved pipe according to the Reynolds number Rₑᵢ of the drilling fluid in the on-site curved pipe, and calculating a shear rate γᵢ of the drilling fluid according to the actual shear stress τwᵢ;
step 4: establishing a plurality of on-site models according to the actual shear stress τwᵢ and the shear rate γᵢ of the drilling fluid;
step 5: determining an optimal on-site model according to correlations between the actual shear stress τwᵢ and predicted shear stresses of the plurality of on-site models; and
step 6: performing on-site measurement on the rheological property of the drilling fluid according to the optimal on-site model.

2. The method for measuring the rheological property of the drilling fluid by using the curved pipe on site according to claim 1, wherein step 1 comprises:
step 11: calculating a friction coefficient f_{ck} of the drilling fluid in an offline curved pipe and a friction coefficient fₛₖ of the drilling fluid in an offline straight pipe, wherein k denotes a number of times the drilling fluid flows through an offline pipe, and k is a positive integer not less than 2;
step 12: establishing a plurality of prediction models according to an actual friction coefficient ratio yₖ, wherein, yₖ=f_{ck}/fₛₖ;
step 13: determining an optimal prediction model according to correlations between the actual friction coefficient ratio yₖ and predicted friction coefficient ratios of the plurality of prediction models; and
step 14: deriving the relationship constants between the friction coefficients of the drilling fluid according to the optimal prediction model.

3. The method for measuring the rheological property of the drilling fluid by using the curved pipe on site according to claim 2, wherein
in step 11, f_{ck} is expressed by formula (1):
$${f}_{c k} = \frac{{d}_{tc 1}}{2 {ρ}_{1} ⋆ {v}_{c k}^{2}} * \frac{Δ {P}_{c k}}{Δ {L}_{c k}} \qquad ( 1 )$$
wherein, d_{tc1} denotes an inner diameter of the offline curved pipe, and has a unit of m;
ρ₁ denotes a density of an offline drilling fluid, and has a unit of kg/m³;
v_{ck} denotes a flow velocity of the drilling fluid at a k-th time in the offline curved pipe, and has a unit of m/s;
ΔP_{ck}/ΔL_{ck} denotes a measured average pressure difference in the offline curved pipe, and has a unit of kPa/m; and
ΔP_{ck} denotes a total pressure difference in a pipe section with a length of ΔL_{ck}, and has a unit of kPa;
in step 11, fₛₖ is expressed by formula (2):
$${f}_{s k} = \frac{{d}_{ts 1}}{2 {ρ}_{1} ⋆ {v}_{sk}^{2}} * \frac{Δ {P}_{sk}}{Δ {L}_{sk}} \qquad ( 2 )$$
wherein, dₜₛ₁ denotes an inner diameter of the offline straight pipe, and has a unit of m;
ρ₁ denotes a density of the drilling fluid, and has a unit of kg/m³;
vₛₖ denotes a flow velocity of the drilling fluid at the k-th time in the offline straight pipe, and has a unit of m/s;
ΔPₛₖ/ΔLₛₖ denotes a measured average pressure difference in the offline straight pipe, and has a unit of kPa/m; and
ΔPₛₖ denotes a total pressure difference in a pipe section with a length of ΔLₛₖ, and has a unit of kPa.

4. The method for measuring the rheological property of the drilling fluid by using the curved pipe on site according to claim 2, wherein the plurality of prediction models at least comprise:
a first prediction model:
ŷ₁ₖ=a*Dₙₖ^{b}+c (3)
a second prediction model:
$${\hat{y}}_{2 k} = 1 + \frac{{a}^{*} {D}_{nk}^{b}}{70 + {D}_{nk}} \qquad ( 4 )$$
a third prediction model:
ŷ₃ₖ=1+a*(log₁₀Dₙₖ)^{b} (5)
wherein
ŷ₁ₖ denotes a predicted friction coefficient of the first prediction model;
ŷ₂ₖ denotes a predicted friction coefficient of the second prediction model;
ŷ₃ₖ denotes a predicted friction coefficient of the third prediction model; and
a, b and c denote the relationship constants between the friction coefficients of the drilling fluid, respectively;
wherein, Dₙₖ denotes a Dean number of the drilling fluid at a k-th time in the offline curved pipe, and is expressed by formula (6):
$${D}_{nk} = \frac{{ρ}_{1}^{*} {{v}_{ck}}^{*} {d}_{tc 1}}{{μ}_{1}} * \sqrt{\frac{{d}_{tc 1}}{{D}_{c 1}}} \qquad ( 6 )$$
wherein
μ₁ denotes a viscosity of an offline drilling fluid, and has a unit of Pa·s; and
v_{ck} denotes a flow velocity of the drilling fluid at the k-th time in the offline curved pipe, and has a unit of m/s.

5. The method for measuring the rheological property of the drilling fluid by using the curved pipe on site according to claim 4, wherein step 13 comprises:
expressing a correlation R₁₁² between the actual friction coefficient ratio yₖ and a predicted friction coefficient ratio of the first prediction model by formula (7):
$${R}_{11}^{2} = 1 - \frac{{\sum }_{k = 1}^{m} {( {y}_{k} - \hat{{y}_{1 k}} )}^{2}}{{\sum }_{k = 1}^{m} {( {y}_{k} - \overline{y} )}^{2}} \qquad ( 7 )$$
expressing a correlation R₁₂² between the actual friction coefficient ratio yₖ and a predicted friction coefficient ratio of the second prediction model by formula (8):
$${R}_{12}^{2} = 1 - \frac{{\sum }_{k = 1}^{m} {( {y}_{k} - \hat{{y}_{2 k}} )}^{2}}{{\sum }_{k = 1}^{m} {( {y}_{k} - \overline{y} )}^{2}} \qquad ( 8 )$$
expressing a correlation R₁₃² between the actual friction coefficient ratio yₖ and a predicted friction coefficient ratio of the third prediction model by formula (9):
$${R}_{13}^{2} = 1 - \frac{{\sum }_{k = 1}^{m} {( {y}_{k} - \hat{{y}_{3 k}} )}^{2}}{{\sum }_{k = 1}^{m} {( {y}_{k} - \overline{y} )}^{2}} \qquad ( 9 )$$
comparing R₁₁², R₁₂² and R₁₃² in terms of magnitude, and selecting a prediction model with a maximum correlation as an optimal prediction model;
wherein
m denotes a number of samples;
yₖ denotes the actual friction coefficient ratio; and
y̅ denotes an average actual friction coefficient ratio.

6. The method for measuring the rheological property of the drilling fluid by using the curved pipe on site according to claim 5, wherein in step 2, f_{ci} is expressed by formula (10):
$${f}_{ci} = \frac{{d}_{tc 2}}{2 {{ρ}_{2}}^{*} {v}_{ci}^{2}} * \frac{Δ {P}_{ci}}{Δ {L}_{ci}} \qquad ( 10 )$$
wherein, d_{tc2} denotes an inner diameter of the on-site curved pipe, and has a unit of m;
ρ₂ denotes a density of an on-site drilling fluid, and has a unit of kg/m³;
v_{ci} denotes a flow velocity of the drilling fluid at an i-th time in the on-site curved pipe, and has a unit of m/s;
ΔP_{ci}/ΔL_{ci} denotes a measured average pressure difference in the on-site curved pipe, and has a unit of kPa/m; and
ΔP_{ci} denotes a total pressure difference in a pipe section with a length of ΔL_{ci}, and has a unit of kPa.

7. The method for measuring the rheological property of the drilling fluid by using the curved pipe on site according to claim 6, wherein in step 2, the Reynolds number Rₑᵢ of the drilling fluid in the on-site curved pipe is calculated as follows:
when an offline model is the first prediction model, the Reynolds number Rₑᵢ of the drilling fluid in the on-site curved pipe satisfies formula (12):
$${f}_{c 1} = \frac{16}{{R}_{ei}} ( {{a}^{*} ( {{R}_{ei}}^{*} \sqrt{\frac{{d}_{tc 2}}{{D}_{c 2}}} )}^{b} + c ) \qquad ( 12 )$$
when the offline model is the second prediction model, the Reynolds number Rₑᵢ of the drilling fluid in the on-site curved pipe satisfies formula (13):
$${f}_{ci} = \frac{16}{{R}_{ei}} * [ 1 + \frac{{{a}^{*} ( {{R}_{ei}}^{*} \sqrt{\frac{{d}_{tc 2}}{{D}_{c 2}}} )}^{b}}{70 + ( {{R}_{ei}}^{*} \sqrt{\frac{{d}_{tc 2}}{{D}_{c 2}}} )} ] , \qquad ( 13 )$$
and
when the offline model is the third prediction model, the Reynolds number Rₑᵢ of the drilling fluid in the on-site curved pipe satisfies formula (14):
$${f}_{ci} = \frac{16}{{R}_{ei}} * [ 1 + {{a}^{*} ( {log}_{10} ( {{R}_{ei}}^{*} \sqrt{\frac{{d}_{tc 2}}{{D}_{c 2}}} ) )}^{b} ] . \qquad ( 14 )$$

8. The method for measuring the rheological property of the drilling fluid by using the curved pipe on site according to claim 7, wherein in step 3, the actual shear stress τwᵢ of the drilling fluid in the on-site curved pipe is expressed by formula (15):
$${τ}_{wi} = \frac{8 {{ρ}_{2}}^{*} {v}_{ci}^{2}}{{R}_{ei}} \qquad ( 15 )$$
wherein
v_{ci} denotes the flow velocity of the drilling fluid at the i-th time in the on-site curved pipe, and has a unit of m/s; and
ρ₂ denotes the density of the on-site drilling fluid, and has a unit of kg/m³;
the shear rate γᵢ of the drilling fluid is expressed by formula (16):
$${γ}_{i} = \frac{{8}^{*} {v}_{ci}}{{d}_{tc 2}} * \frac{{3}^{*} {N}_{i} + 1}{{4}^{*} {N}_{i}} \qquad ( 16 )$$
wherein, N is expressed by formula (17):
$${N}_{i} = \frac{d ( ln {τ}_{{w}_{i}} )}{d ( ln \frac{{8}^{*} {v}_{ci}}{{d}_{tc 2}} )} . \qquad ( 17 )$$

9. The method for measuring the rheological property of the drilling fluid by using the curved pipe on site according to claim 8, wherein the plurality of on-site models at least comprise:
a first on-site model:
\hat{τ}w₁ᵢ=YP+PV*γᵢ (18)
a second on-site model:
\hat{τ}w₂ᵢ=K*γᵢⁿ (19),
a third on-site model:
\hat{τ}w₂ᵢ=τ₀+K*γᵢⁿ (20),
wherein
YP denotes a yield strength of the on-site drilling fluid, and has a unit of Pa;
PV denotes a plastic viscosity of the on-site drilling fluid, and has a unit of Pa·s;
n denotes a fluidity index of the on-site drilling fluid, and is dimensionless;
K denotes a consistency coefficient of the on-site drilling fluid, and has a unit of Pa·s\hat{ }n; and
τ₀ denotes a dynamic shear stress of the on-site drilling fluid, and has a unit of Pa.

10. The method for measuring the rheological property of the drilling fluid by using the curved pipe on site according to claim 9, wherein step 5 comprises:
expressing a correlation R₂₁² between the actual shear stress τwᵢ and a predicted shear stress of the first on-site model by formula (21):
$${R}_{21}^{2} = 1 - \frac{{\sum }_{i = 1}^{m} {( τ {w}_{i} - \hat{τ {w}_{1 i}} )}^{2}}{{\sum }_{i = 1}^{m} {( τ {w}_{i} - \overline{τ w} )}^{2}} \qquad ( 21 )$$
expressing a correlation R₂₂² between the actual shear stress τwᵢ and a predicted shear stress of the second on-site model by formula (22):
$${R}_{22}^{2} = 1 - \frac{{\sum }_{i = 1}^{m} {( τ {w}_{i} - \hat{τ {w}_{2 i}} )}^{2}}{{\sum }_{i = 1}^{m} {( τ {w}_{i} - \overline{τ w} )}^{2}} \qquad ( 22 )$$
expressing a correlation R₂₃² between the actual shear stress τwᵢ and a predicted shear stress of the third on-site model by formula (23):
$${R}_{23}^{2} = 1 - \frac{{\sum }_{i = 1}^{m} {( τ {w}_{i} - \hat{τ {w}_{3 i}} )}^{2}}{{\sum }_{i = 1}^{m} {( τ {w}_{i} - \overline{τ w} )}^{2}} \qquad ( 23 )$$
comparing R₂₁², R₂₂² and R₂₃² in terms of magnitude, and selecting an on-site model with a maximum correlation as a final model;
wherein
m denotes a number of samples;
τwᵢ denotes the actual shear stress; and
τ̅w̅ denotes an average actual shear stress.
